(19) United States
(12) Patent Application Publication
(10) Pub. No.: US 2016/0310041 A1
(43) Pub. Date: Oct. 27, 2016

(54) GUIDEWIRE WITH NAVIGATION SENSOR

(71) Applicant: Acclarent, Inc., Menlo Park, CA (US)

(72) Inventors: Thomas R. Jenkins, Alameda, CA (US); Randy J. Kesten, Mountain View, CA (US); Rohit Girotra, Mountain View, CA (US)

(21) Appl. No.: 14/835,108

(22) Filed: Aug. 25, 2015

Related U.S. Application Data
(60) Provisional application No. 62/150,954, filed on Apr. 22, 2015.

(51) Int. Cl. A61B 5/06; A61B 5/00

(52) U.S. Cl. CPC A61B 5/062; A61B 5/6851; A61M 2025/09083

(57) ABSTRACT

An apparatus includes an outer coil, a navigation coil, and a distal tip member. The outer coil defines an interior region bounded by inner diameter. The navigation coil is positioned distal to the distal end of the outer coil. The navigation coil is configured to generate a signal in response to movement of the navigation coil within an electromagnetic field. The distal tip member is positioned distal to the navigation coil, such that the navigation coil is longitudinally interposed between the distal tip member and the distal end of the outer coil. The apparatus may be used in combination with a navigation system to provide navigation of paranasal sinus cavities and other structures within a patient.

### PRIORITY

This application claims priority to U.S. Provisional Patent App. No. 62/150,954, entitled “Guidewire with Navigation Sensor,” filed Apr. 22, 2015, the disclosure of which is incorporated by reference herein.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Menlo Park, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein. An example of such an endoscope is the Acclarent Cyclops™ Multi-Angle Endoscope by Acclarent, Inc. of Menlo Park, Calif.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pub. No. 2012/0078118, entitled “Sinus Illumination Lightwire Device,” published Mar. 29, 2012, the disclosure of which is incorporated by reference herein. An example of such an illuminating guidewire is the Relieva Luma Sentry™ Sinus Illumination System by Acclarent, Inc. of Menlo Park, Calif.

It may be desirable to provide easily controlled inflation/deflation of a balloon in dilation procedures, including procedures that will be performed only by a single operator. While several systems and methods have been made and used to inflate an inflatable member such as a dilation balloon, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side elevational view of an exemplary dilation catheter system;

FIG. 2A depicts a side elevational view of an exemplary illuminating guidewire of the dilation catheter system of FIG. 1;

FIG. 2B depicts a side elevational view of an exemplary guide catheter of the dilation catheter system of FIG. 1;

FIG. 2C depicts a side elevational view of an exemplary dilation catheter of the dilation catheter system of FIG. 1;

FIG. 3 depicts a detailed side elevational view of the illuminating guide wire of FIG. 2A;

FIG. 4 depicts a detailed side cross-sectional view of the illuminating guidewire of FIG. 2A;

FIG. 5 depicts a perspective view of an exemplary endoscope suitable for use with the dilation catheter system of FIG. 1;

FIG. 6 depicts a side elevational view of the distal end of the endoscope of FIG. 5, showing an exemplary range of viewing angles;

FIG. 7A depicts a front view of the guide catheter of FIG. 2B positioned adjacent an ostium of the maxillary sinus;

FIG. 7B depicts a front view of the guide catheter of FIG. 2B positioned adjacent an ostium of the maxillary sinus, with the dilation catheter of FIG. 2C and the illuminating guidewire of FIG. 2A positioned in the guide catheter and a distal portion of the guidewire positioned in the maxillary sinus;

FIG. 7C depicts a front view of the guide catheter of FIG. 2B positioned adjacent an ostium of the maxillary sinus, with the illuminating guidewire of FIG. 2A translated further distally relative to the guide catheter and into the maxillary sinus;

FIG. 7D depicts a front view of the guide catheter of FIG. 2B positioned adjacent an ostium of the maxillary sinus, with the dilation catheter of FIG. 2C translated distally relative to the guide catheter along the illuminating guidewire of FIG. 2A so as to position a balloon of the dilation catheter within the ostium;

FIG. 7E depicts a front view of an ostium of the maxillary sinus, with the ostium having been enlarged by inflation of the balloon of FIG. 7D;

FIG. 8 depicts a schematic perspective view of a modified version of the dilation catheter system of FIG. 1 being used in conjunction with an exemplary image guided navigation system;

FIG. 9 depicts a cross-sectional side view of the distal end of an exemplary guidewire that may be incorporated into the modified dilation catheter system of FIG. 8;

FIG. 10 depicts a cross-sectional side view of the distal end of another exemplary guidewire that may be incorporated into the modified dilation catheter system of FIG. 8;

FIG. 11 depicts a cross-sectional side view of the distal end of another exemplary guidewire that may be incorporated into the modified dilation catheter system of FIG. 8;

FIG. 12 depicts a cross-sectional side view of the distal end of another exemplary guidewire that may be incorporated into the modified dilation catheter system of FIG. 8;

FIG. 13 depicts a cross-sectional side view of the distal end of another exemplary guidewire that may be incorporated into the modified dilation catheter system of FIG. 8;

FIG. 14 depicts a cross-sectional side view of the distal end of another exemplary guidewire that may be incorporated into the modified dilation catheter system of FIG. 8;

FIG. 15 depicts a perspective view of the distal end of another exemplary guidewire that may be incorporated into the modified dilation catheter system of FIG. 8;

FIG. 16 depicts a cross-sectional side view of the distal end of the guidewire of FIG. 15;

FIG. 17 depicts a perspective view of the distal end of another exemplary guidewire that may be incorporated into the modified dilation catheter system of FIG. 8; and

FIG. 18 depicts a cross-sectional side view of the distal end of the guidewire of FIG. 17.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Dilation Catheter System

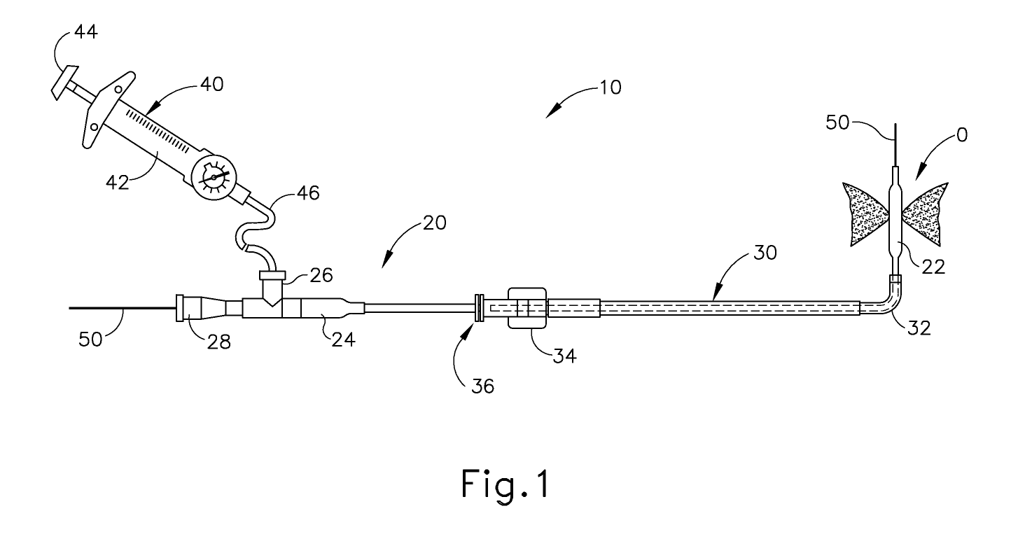

FIG. 1 shows an exemplary dilation catheter system (10) that may be used to dilate the ostium of a paranasal sinus; or to dilate some other anatomical passageway (e.g., within the ear, nose, or throat, etc.). Dilation catheter system (10) of this example comprises a dilation catheter (20), a guide catheter (30), an inflator (40), and a guidewire (50). By way of example only, dilation catheter system (10) may be configured in accordance with at least some of the teachings of U.S. Patent Pub. No. 2011/0004057, the disclosure of which is incorporated by reference herein. In some versions, at least part of dilation catheter system (10) is configured similar to the Relieva® Spin Balloon Sinuplasty™ System by Acclarent, Inc. of Menlo Park, Calif.

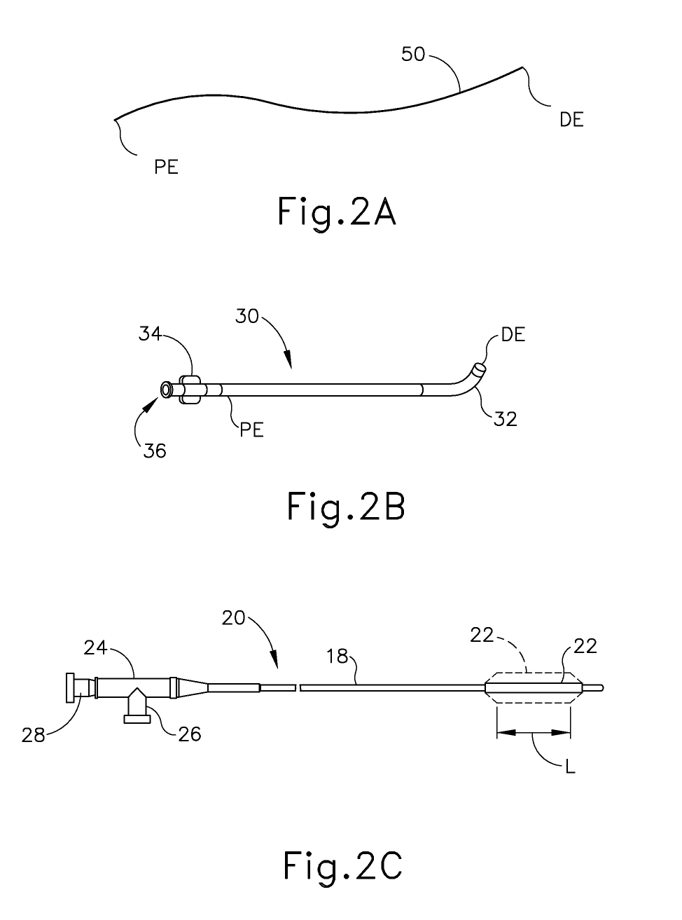

As best seen in FIG. 2C, the distal end (DE) of dilation catheter (20) includes an inflatable dilator (22). The proximal end (PE) of dilation catheter (20) includes a grip (24), which has a lateral port (26) and an open proximal end (28). A hollow-elongate shaft (18) extends distally from grip. Dilation catheter (20) includes a first lumen (not shown) formed within shaft (18) that provides fluid communication between lateral port (26) and the interior of dilator (22). Dilator catheter (20) also includes a second lumen (not shown) formed within shaft (18) that extends from open proximal end (28) to an open distal end that is distal to dilator (22). This second lumen is configured to slidably receive guidewire (50). The first and second lumens of dilator catheter (20) are fluidly isolated from each other. Thus, dilator (22) may be selectively inflated and deflated by communicating fluid along the first lumen via lateral port (26) while guidewire (50) is positioned within the second lumen. In some versions, dilator catheter (20) is configured similar to the Relieva Ultirra™ Sinus Balloon Catheter by Acclarent, Inc. of Menlo Park, Calif. In some other versions, dilator catheter (20) is configured similar to the Relieva Solo Pro™ Sinus Balloon Catheter by Acclarent, Inc. of Menlo Park, Calif. Other suitable forms that dilator catheter (20) may take will be apparent to those of ordinary skill in the art in view of the teachings herein.

As best seen in FIG. 2B, guide catheter (30) of the present example includes a bent distal portion (32) at its distal end (DE) and a grip (34) at its proximal end (PE). Grip (34) has an open proximal end (36). Guide catheter (30) defines a lumen that is configured to slidably receive dilation catheter (20), such that guide catheter (30) may guide dilator (22) out through bent distal end (32). In some versions, guide catheter (30) is configured similar to the Relieva Flex™ Sinus Guide Catheter by Acclarent, Inc. of Menlo Park, Calif. Other suitable forms that guide catheter (30) may take will be apparent to those of ordinary skill in the art in view of the teachings herein.

Referring back to FIG. 1, inflator (40) of the present example comprises a barrel (42) that is configured to hold fluid and a plunger (44) that is configured to reciprocate relative to barrel (42) to selectively discharge fluid from (or draw fluid into) barrel (42). Barrel (42) is fluidly coupled with lateral port (26) via a flexible tube (46). Thus, inflator (40) is operable to add fluid to dilator (22) or withdraw fluid from dilator (22) by translating plunger (44) relative to barrel (42). In the present example, the fluid communicated by inflator (40) comprises saline, though it should be understood that any other suitable fluid may be used. There are various ways in which inflator (40) may be filled with fluid (e.g., saline, etc.). By way of example only, before flexible tube (46) is coupled with lateral port (26), the distal end of flexible tube (46) may be placed in a reservoir containing the fluid. Plunger (44) may then be retracted from a distal position to a proximal position to draw the fluid into barrel (42). Inflator (40) may then be held in an upright position, with the distal end of barrel (42) pointing upwardly, and plunger (44) may then be advanced to an intermediate or slightly distal position to purge any air from barrel (42). The distal end of flexible tube (46) may then be coupled with lateral port (26). In some versions, inflator (40) is constructed and operable in accordance with at least some of the teachings of U.S. Pub. No. 2014/0074141, entitled “Inflator for Dilation of Anatomical Passageway,” published Mar. 13, 2014, the disclosure of which is incorporated by reference herein.

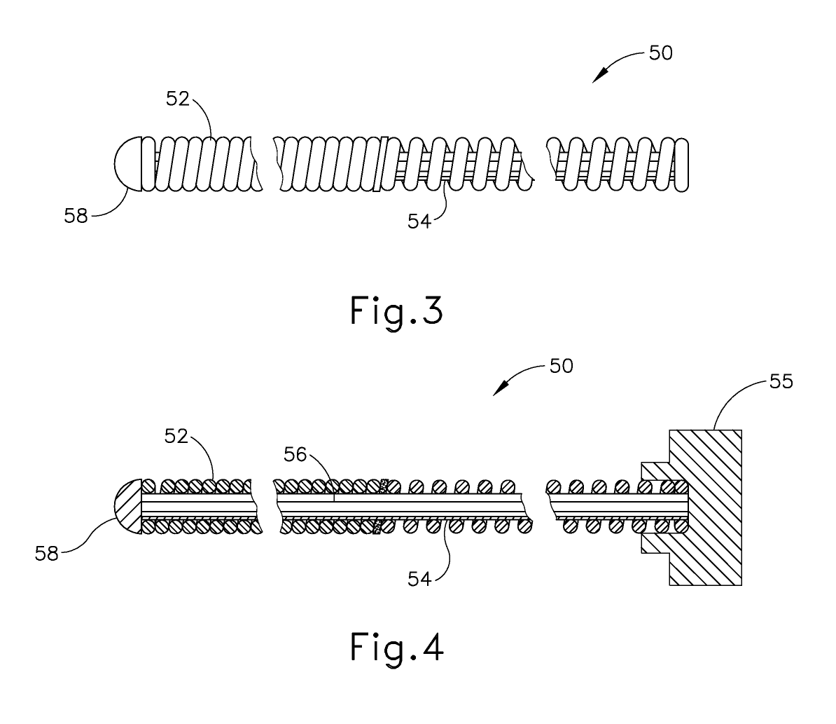

As shown in FIGS. 2A, 3, and 4, guidewire (50) of the present example comprises a coil (52) positioned about a core wire (54). An illumination fiber (56) extends along the interior of core wire (54) and terminates in an atraumatic lens (58). A connector (55) at the proximal end of guidewire (50) enables optical coupling between illumination fiber (56) and a light source (not shown). Illumination fiber (56) may comprise one or more optical fibers. Lens (58) is configured to project light when illumination fiber (56) is illuminated by the light source, such that illumination fiber (56) transmits light from the light source to the lens (58). In some versions, the distal end of guidewire (50) is more flexible than the proximal end of guidewire (50). Guidewire (50) has a length enabling the distal end of guidewire (50) to be positioned distal to dilator (22) while the proximal end of guidewire (50) is positioned proximal to grip (24). Guidewire (50) may include indicia along at least part of its length (e.g., the proximal portion) to provide the operator with visual feedback indicating the depth of insertion of guidewire (50) relative to dilation catheter (20). By way of example only, guidewire (50) may be configured in accordance with at least some of the teachings of U.S. Pub. No. 2012/0078118, the disclosure of which is incorporated by reference herein. In some versions, guidewire (50) is configured similar to the Relieva Luma Sentry™ Sinus Illumination System by Acclarent, Inc. of Menlo Park, Calif. Other suitable forms that guidewire (50) may take will be apparent to those of ordinary skill in the art in view of the teachings herein.

II. Overview of Exemplary Endoscope

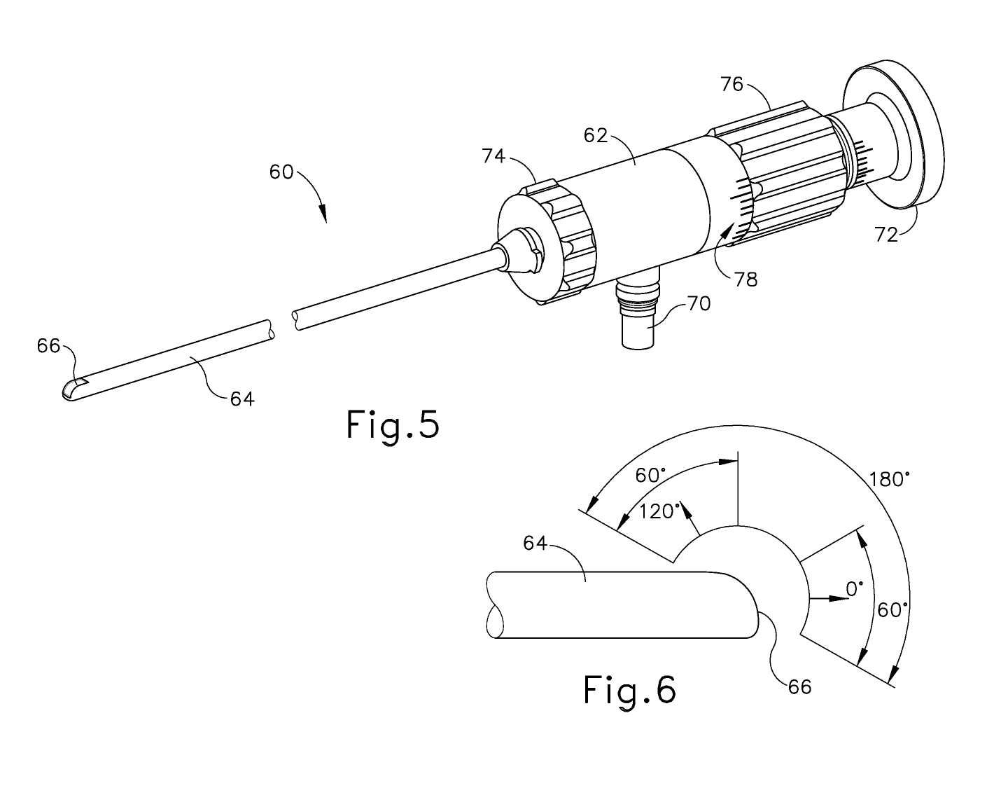

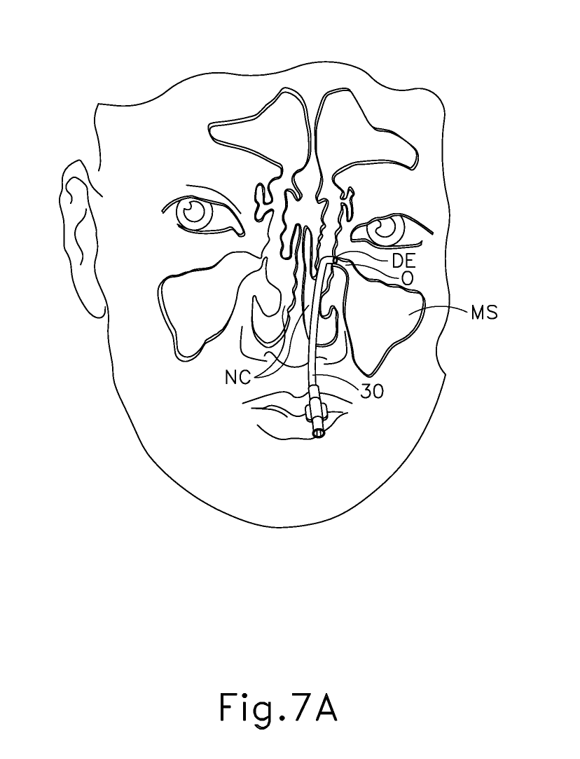

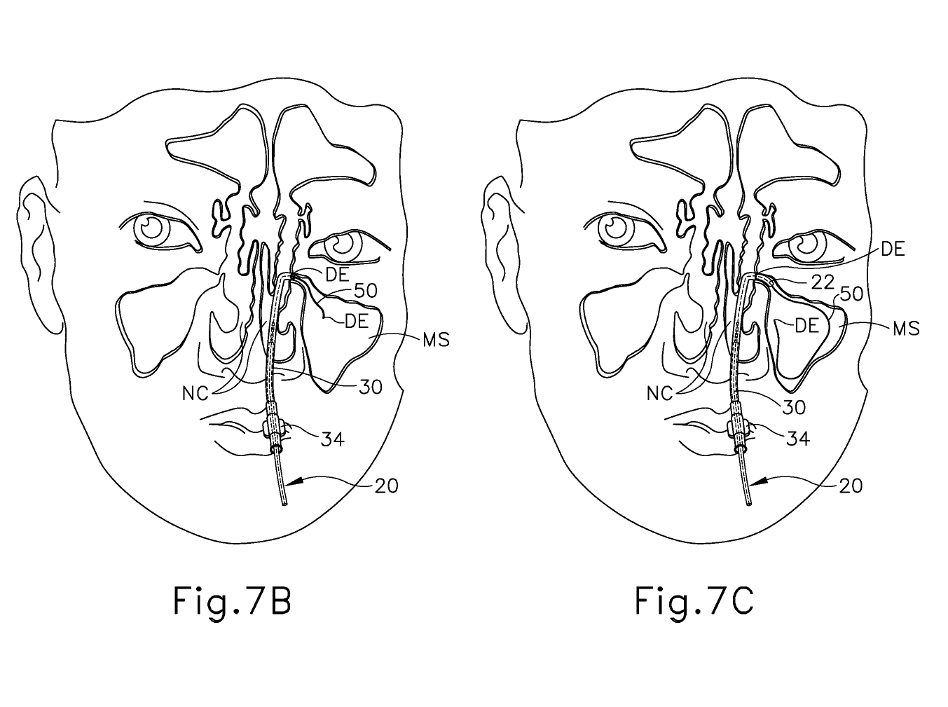

As noted above, an endoscope (60) may be used to provide visualization within an anatomical passageway (e.g., within the nasal cavity, etc.) during a process of using dilation catheter system (10). As shown in FIGS. 4-5, endoscope of the present example comprises a body (62) and a rigid shaft (64) extending distally from body (62). The distal end of shaft (64) includes a curved transparent window (66). A plurality of rod lenses and light transmitting fibers may extend along the length of shaft (64). A lens is positioned at the distal end of the rod lenses and a swing prism is positioned between the lens and window (66). The swing prism is pivotable about an axis that is transverse to the longitudinal axis of shaft (64). The swing prism defines a line of sight that pivots with the swing prism. The line of sight defines a viewing angle relative to the longitudinal axis of shaft (64). This line of sight may pivot from approximately 0 degrees to approximately 120 degrees, from approximately 10 degrees to approximately 90 degrees, or within any other suitable range. The swing prism and window (66) also provide a field of view spanning approximately 60 degrees (with the line of sight centered in the field of view). Thus, the field of view enables a viewing range spanning approximately 180 degrees, approximately 140 degrees, or any other range, based on the pivot range of the swing prism. Of course, all of these values are mere examples.

Body (62) of the present example includes a light post (70), an eyepiece (72), a rotation dial (74), and a pivot dial (76). Light post (70) is in communication with the light transmitting fibers in shaft (64) and is configured to couple with a source of light, to thereby illuminate the site in the patient distal to window (66). Eyepiece (72) is configured to provide visualization of the view captured through window (66) via the optics of endoscope (60). It should be understood that a visualization system (e.g., camera and display screen, etc.) may be coupled with eyepiece (72) to provide visualization of the view captured through window (66) via the optics of endoscope (60). Rotation dial (74) is configured to rotate shaft (64) relative to body (62) about the longitudinal axis of shaft (64). It should be understood that such rotation may be carried out even while the swing prism is pivoted such that the line of sight is non-parallel with the longitudinal axis of shaft (64). Pivot dial (76) is coupled with the swing prism and is thereby operable to pivot the swing prism about the transverse pivot axis. Indicia (78) on body (62) provide visual feedback indicating the viewing angle. Various suitable components and arrangements that may be used to couple rotation dial (74) with the swing prism will be apparent to those of ordinary skill in the art in view of the teachings herein. By way of example only, endoscope (60) may be configured in accordance with at least some of the teachings of U.S. Pub. No. 2010/0030031, the disclosure of which is incorporated by reference herein. In some versions, endoscope (60) is configured similar to the Acclarent Cyclops™ Multi-Angle Endoscope by Acclarent, Inc. of Menlo Park, Calif. Other suitable forms that endoscope (60) may take will be apparent to those of ordinary skill in the art in view of the teachings herein

III. Exemplary Method for Dilating the Ostium of a Maxillary Sinus

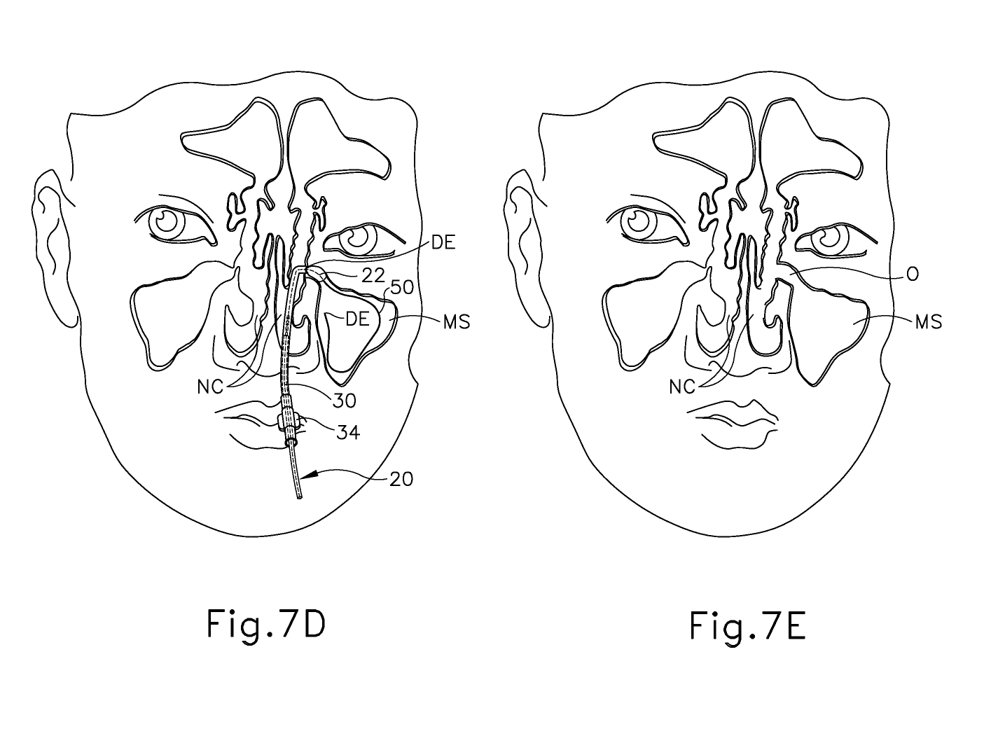

FIGS. 7A-7E show an exemplary method for using dilation catheter system (10) discussed above to dilate a sinus ostium (O) of a maxillary sinus (MS) of a patient. While the present example is being provided in the context of dilating a sinus ostium (O) of a maxillary sinus (MS), it should be understood that dilation catheter system (10) may be used in various other procedures. By way of example only, dilation catheter system (10) and variations thereof may be used to dilate a Eustachian tube, a larynx, a choana, a sphenoid sinus ostium, one or more openings associated with one or more ethmoid sinus air cells, the frontal recess, and/or other passageways associated with paranasal sinuses. Other suitable ways in which dilation catheter system (10) may be used will be apparent to those of ordinary skill in the art in view of the teachings herein.

In the procedure of the present example, guide catheter (30) may be inserted transnasally and advanced through the nasal cavity (NC) to a position within or near the targeted anatomical passageway to be dilated, the sinus ostium (O), as shown in FIG. 7A. Inflatable dilator (22) and the distal end of guidewire (50) may be positioned within or proximal to bent distal end (32) of guide catheter (30) at this stage. This positioning of guide catheter (30) may be verified endoscopically with an endoscope such as endoscope (60) described above and/or by direct visualization, radiography, and/or by any other suitable method. After guide catheter (30) has been positioned, the operator may advance guidewire (50) distally through guide catheter (30) such that a distal portion of the guidewire (50) passes through the ostium (O) of the maxillary sinus (MS) and into the cavity of the maxillary sinus (MS) as shown in FIGS. 7B and 7C. The operator may illuminate illumination fiber (56) and lens (58), which may provide transcutaneous illumination through the patient's face to enable the operator to visually confirm positioning of the distal end of guidewire (50) in the maxillary sinus (MS) with relative ease.

As shown in FIG. 7C, with guide catheter (30) and guidewire (50) suitably positioned, dilation catheter (20) is advanced along guidewire (50) and through bent distal end (32) of guide catheter (30), with dilator (22) in a non-dilated state until dilator (22) is positioned within the ostium (O) of the maxillary sinus (MS) (or some other targeted anatomical passageway). After dilator (22) has been positioned within the ostium (O), dilator (22) may be inflated, thereby dilating the ostium (O), as shown in FIG. 7D. To inflate dilator (22), plunger (44) may be actuated to push saline from barrel (42) of inflator (40) through dilation catheter (20) into dilator (22). The transfer of fluid expands dilator (22) to an expanded state to open or dilate the ostium (O), such as by remodeling the bone, etc., forming ostium (O). By way of example only, dilator (22) may be inflated to a volume sized to achieve about 10 to about 12 atmospheres. Dilator (22) may be held at this volume for a few seconds to sufficiently open the ostium (O) (or other targeted anatomical passageway). Dilator (22) may then be returned to a non-expanded state by reversing plunger (44) of inflator (40) to bring the saline back to inflator (40). Dilator (22) may be repeatedly inflated and deflated in different ostia and/or other targeted anatomical passageways. Thereafter, dilation catheter (20), guidewire (50), and guide catheter (30) may be removed from the patient as shown in FIG. 7E.

In some instances, it may be desirable to irrigate the sinus and paranasal cavity after dilation catheter (20) has been used to dilate the ostium (O). Such irrigation may be performed to flush out blood, etc. that may be present after the dilation procedure. For example, in some cases, guide catheter (30) may be allowed to remain in place after removal of guidewire (50) and dilation catheter (20) and a lavage fluid, other substance, or one or more other devices (e.g., lavage catheters, balloon catheters, cutting balloons, cutters, chompers, rotating cutters, rotating drills, rotating blades, sequential dilators, tapered dilators, punches, dissectors, burs, non-inflating mechanically expandable members, high frequency mechanical vibrators, dilating stents and radiofrequency ablation devices, microwave ablation devices, laser devices, snares, biopsy tools, scopes, and devices that deliver diagnostic or therapeutic agents) may be passed through guide catheter (30) for further treatment of the condition. By way of example only, irrigation may be carried out in accordance with at least some of the teachings of U.S. Pat. No. 7,630,676, entitled “Methods, Devices and Systems for Treatment and/or Diagnosis of Disorders of the Ear, Nose and Throat,” issued Dec. 8, 2009, the disclosure of which is incorporated by reference herein. An example of an irrigation catheter that may be fed through guide catheter (30) to reach the irrigation site after removal of dilation catheter (20) is the Relieva Vortex® Sinus Irrigation Catheter by Acclarent, Inc. of Menlo Park, Calif. Another example of an irrigation catheter that may be fed through guide catheter (30) to reach the irrigation site after removal of dilation catheter (20) is the Relieva Ultirra® Sinus Irrigation Catheter by Acclarent, Inc. of Menlo Park, Calif. Of course, irrigation may be provided in the absence of a dilation procedure; and a dilation procedure may be completed without also including irrigation.

IV. Exemplary Image Guided Navigation System

Image-guided surgery (IGS) is a technique wherein a computer is used to obtain a real-time correlation of the location of an instrument that has been inserted into a patient's body to a set of preoperatively obtained images (e.g., a CT or MRI scan, 3-D map, etc.) so as to superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot) showing the real time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

Examples of electromagnetic IGS systems that may be used in ENT and sinus surgery include the InstaTrak ENT™ systems available from GE Medical Systems, Salt Lake City, Utah. Other examples of electromagnetic image guidance systems that may be modified for use in accordance with the present disclosure include but are not limited to the CARTO® 3 System by Biosense-Webster, Inc., of Diamond Bar, Calif.; systems available from Surgical Navigation Technologies, Inc., of Louisville, Colo.; and systems available from Calypso Medical Technologies, Inc., of Seattle, Wash.

When applied to functional endoscopic sinus surgery (FESS), balloon sinuplasty, and/or other ENT procedures, the use of image guidance systems allows the surgeon to achieve more precise movement and positioning of the surgical instruments than can be achieved by viewing through an endoscope alone. This is so because a typical endoscopic image is a spatially limited, 2 dimensional, line-of-sight view. The use of image guidance systems provides a real time, 3 dimensional view of all of the anatomy surrounding the operative field, not just that which is actually visible in the spatially limited, 2 dimensional, direct line-of-sight endoscopic view. As a result, image guidance systems may be particularly useful during performance of FESS, balloon sinuplasty, and/or other ENT procedures, especially in cases where normal anatomical landmarks are not present or are difficult to visualize endoscopically.

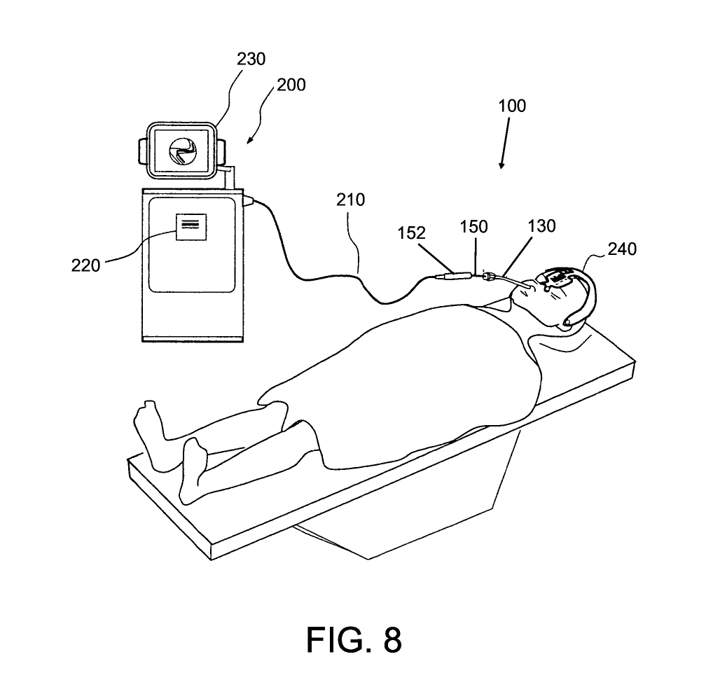

FIG. 8 shows a modified dilation catheter system (100) in combination with an exemplary image guidance system (200). Dilation catheter system (100) comprises a guide catheter (130) with a guidewire (150) slidably disposed therein. Guide catheter (130) may be constructed and operable just like guide catheter (30) described above, such that further details will not be provided here. It should also be understood that, while not shown in FIG. 8, dilation catheter system (100) may also include a dilation catheter that is constructed and operable just like dilation catheter (20) described above. The dilation catheter may be slid along guidewire (150) and through guide catheter (130) as described above.

Guidewire (150) of this example is substantially similar to guidewire (50) described above, except that guidewire (150) of this example is particularly configured to operate in conjunction with navigation system (200). In particular, guidewire (150) includes a connector hub (152) that is configured to couple with a cable (210) of image guidance system (200). The distal end of guidewire (150) includes a coil (not shown) that is in communication with one or more electrical conduits that extend along the length of guidewire (150). When the coil is positioned within an electromagnetic field, movement of the coil within that magnetic field may generate electrical current in the coil, and this electrical current may be communicated along the electrical conduit(s) in guidewire (150) and further along cable (210) via connector hub (152). This phenomenon may enable image guidance system (200) to determine the location of the distal end of guidewire (150) within a three dimensional space as will be described in greater detail below.

While guidewire (150) only has one coil in the present example, it should be understood that guidewire (150) may have two or more coils. Moreover, guidewire (150) may have some other kind of position sensing component that does not necessarily constitute a coil. It should be understood that the distal end of guidewire (150) may be constructed in numerous ways. Several merely illustrative examples of ways in which the distal end of guidewire (150) may be constructed will be described in greater detail below.

Image guidance system (200) of this example further comprises a computer (220), a video display monitor (230), and a field emitting assembly (240). Field emitting assembly (240) is operable to generate an electromagnetic field around the head of the patient. By way of example only, field emitting assembly (240) may comprise a set of coils. Various suitable components that may be used to form and drive field emitting assembly (240) will be apparent to those of ordinary skill in the art in view of the teachings herein. While field emitting assembly (240) is shown as being part of a headset worn by the patient in FIG. 8, it should be understood that field emitting assembly (240) may be positioned at any other suitable location.

Computer (220) includes hardware and software that is configured to drive field emitting assembly (240) and process signals generated by the coil(s) of guidewire (150). In particular, as guidewire (150) is moved within the field generated by field emitting assembly (240), the coil(s) generates position related signals and these signals are communicated to computer (220) via connector hub (152) and cable (210). A processor in computer (220) executes an algorithm to calculate location coordinates of the distal end of guidewire (150) from the position related signals of the coil(s) in guidewire (150). Computer (220) is further operable to provide video in real time via video display monitor (230), showing the position of the distal end of guidewire (150) in relation to a three dimensional model of the anatomy within and adjacent to the patient's nasal cavity.

In some instances, guidewire (150) is used to generate a three dimensional model of the anatomy within and adjacent to the patient's nasal cavity; in addition to being used to provide navigation for dilation catheter system (100) within the patient's nasal cavity. Alternatively, any other suitable device may be used to generate a three dimensional model of the anatomy within and adjacent to the patient's nasal cavity before guidewire (150) is used to provide navigation for dilation catheter system (100) within the patient's nasal cavity. By way of example only, a model of this anatomy may be generated in accordance with at least some of the teachings of U.S. patent application Ser. No. 14/825,551, entitled “System and Method to Map Structures of Nasal Cavity,” filed Aug. 13, 2015, the disclosure of which is incorporated by reference herein. Still other suitable ways in which a three dimensional model of the anatomy within and adjacent to the patient's nasal cavity may be generated will be apparent to those of ordinary skill in the art in view of the teachings herein. It should also be understood that, regardless of how or where the three dimensional model of the anatomy within and adjacent to the patient's nasal cavity is generated, the model may be stored on computer (220). Computer (220) may thus render images of at least a portion of the model via video display monitor (230) and further render real-time video images of the position of guidewire (150) in relation to the model via video display monitor (230).

By way of example only, dilation catheter system (100) and/or image guidance system (200) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 8,190,389, entitled “Adapter for Attaching Electromagnetic Image Guidance Components to a Medical Device,” issued May 29, 2012, the disclosure of which is incorporated by reference herein; U.S. Pat. No. 8,123,722, entitled “Devices, Systems and Methods for Treating Disorders of the Ear, Nose and Throat,” issued Feb. 28, 2012, the disclosure of which is incorporated by reference herein; and U.S. Pat. No. 7,720,521, entitled “Methods and Devices for Performing Procedures within the Ear, Nose, Throat and Paranasal Sinuses,” issued May 18, 2010, the disclosure of which is incorporated by reference herein.

By way of further example only, dilation catheter system (100) and/or image guidance system (200) may be constructed and operable in accordance with at least some of the teachings of U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pat. Pub. No. 2014/0200444, entitled “Guidewires for Performing Image Guided Procedures,” published Jul. 17, 2014, the disclosure of which is incorporated by reference herein; U.S. Pat. Pub. No. 2012/0245456, entitled “Adapter for Attaching Electromagnetic Image Guidance Components to a Medical Device,” published Sep. 27, 2012, the disclosure of which is incorporated by reference herein; U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein; U.S. Pat. Pub. No. 2008/0281156, entitled “Methods and Apparatus for Treating Disorders of the Ear Nose and Throat,” published Nov. 13, 2008, the disclosure of which is incorporated by reference herein; and U.S. Pat. Pub. No. 2007/0208252, entitled “Systems and Methods for Performing Image Guided Procedures within the Ear, Nose, Throat and Paranasal Sinuses,” published Sep. 6, 2007, the disclosure of which is incorporated by reference herein.

It should be understood from the foregoing that the combination of dilation catheter system (100) and image guidance system (200) may be used to perform image guided dilation procedures within the ostia of the various paranasal sinuses, within the frontal recess, within the Eustachian tube, and/or within other passageways associated with the ear, nose, and throat. For instance, the combination of dilation catheter system (100) and image guidance system (200) may be used to perform the dilation of the sinus ostium (O) of the maxillary sinus (MS) as shown in FIGS. 7A-7E and described above. Even in instances where an endoscope such as endoscope (60) is used to provide some degree of visualization within the nasal cavity, the addition of the coil sensor in guidewire (150) and the imaging functionality provided through image guidance system (200) may further enhance the experience of the operator by effectively providing further visualization of anatomical regions that cannot be viewed through endoscope (60). Furthermore, the imaging functionality provided through image guidance system (200) may provide further feedback to the operator indicating the positioning of guidewire (150) within the patient with a degree of precision that could not be obtained using a conventional guidewire (50). Other potential benefits and functionalities that may be obtained through using the combination of dilation catheter system (100) and image guidance system (200) will be apparent to those of ordinary skill in the art in view of the teachings herein.

V. Exemplary Alternative Navigation Guidewire Configurations

As noted above, the distal end of guidewire (150) has a coil that is cooperates with image guidance system (200) to provide signals indicative of the position of the distal end of guidewire (150) in the patient, in real time. Such a coil may be integrated into the distal end of guidewire (150) in numerous different ways. Some ways in which such a coil may be integrated into the distal end of guidewire (150) are described in one or more references that are cited herein. Other examples of how a coil may be integrated into the distal end of guidewire (150) are described in greater detail below. It should also be understood that, in addition to incorporating the teachings below, the various exemplary guidewires described below may also incorporate the teachings of U.S. Pub. No. 2012/0078118, the disclosure of which is incorporated by reference herein. Any of the guidewires described below may be readily incorporated into dilation catheter system (10, 100) in place of guidewire (50, 150).

A. Exemplary Navigation Guidewire with Coil Sensor Inside Distal Tip Member

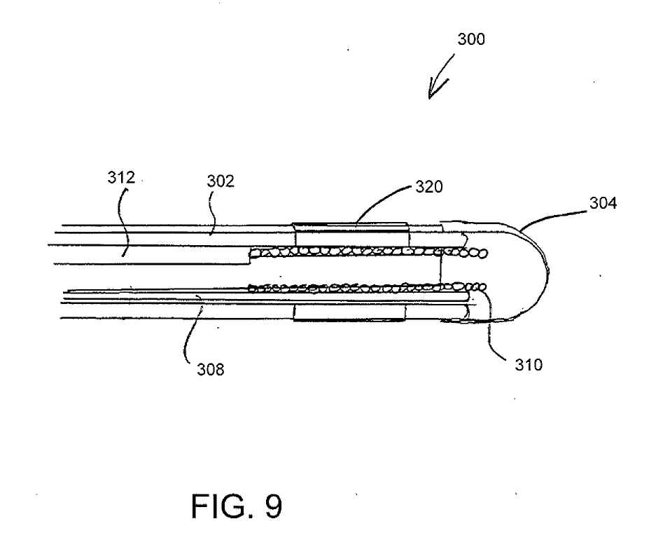

FIG. 9 shows an exemplary guidewire (300) that may be incorporated into dilation catheter system (100) for use with image guidance system (200). Except as otherwise noted herein, guidewire (300) may be constructed and operable just like guidewires (50, 150) described above. Guidewire (300) of this example comprises an outer coil (302), a distal tip member (304), a core wire (308), a navigation coil (310), a navigation cable (312), and a solder joint (320). Outer coil (302) extends along the length of guidewire (300) and contains core wire (308), a proximal portion of navigation coil (310), and navigation cable (312). Outer coil (302) may be constructed in accordance with any suitable conventional guidewire outer coil.

Distal tip member (304) has an atraumatic dome shape and is secured to the distal end of outer coil (302). By way of example only, distal tip member (304) may be formed of an optically transmissive polymeric material and may be secured to the distal end of outer coil (302) using an interference fit, welding, adhesive, or using any other suitable techniques. As another merely illustrative example, distal tip member (304) may be formed by an optically transmissive adhesive that is applied to the distal end of outer coil (302) and then cured. It should also be understood that distal tip member (304) may be configured and operable like lens (58) described above. Other suitable ways in which distal tip member (304) may be configured and operable will be apparent to those of ordinary skill in the art in view of the teachings herein.

In some versions, the distal end of an optical fiber (not shown) is optically coupled with distal tip member (304). The proximal end of the optical fiber is configured to couple with a light source. Various suitable ways in which an optical fiber may be coupled with a light source will be apparent to those of ordinary skill in the art in view of the teachings herein. The optical fiber is configured to provide a path for communication of light from the light source to distal tip member (304), such that distal tip member (304) can emit light generated by the light source. By way of example only one or more optical fibers may run alongside the outer diameter defined by navigation coil (310) in order to reach distal tip member (304). As another merely illustrative example, one or more optical fibers may terminate in the sidewall of outer coil (302) at a location just proximal to navigation coil (310), such that the one or more optical fibers may emit light through the sidewall of outer coil (302). In versions where guidewire (300) includes an optical fiber, it should be understood that any suitable number of optical fibers may be used. Various suitable ways in which guidewire (300) may incorporate one or more optical fibers will be apparent to those of ordinary skill in the art in view of the teachings herein. It should also be understood that guidewire (300) may simply lack any optical fibers.

Core wire (308) is configured to provide additional structural integrity to outer coil (302). In the present example, the proximal end of core wire (308) is fixedly secured to the proximal end of outer coil (302), while the distal end of core wire (308) is fixedly secured to the distal end of outer coil (302). Core wire (308) thus prevents or restricts longitudinal stretching of outer coil (302). Various suitable materials and configurations that may be used to form core wire (308) will be apparent to those of ordinary skill in the art in view of the teachings herein.

Navigation coil (310) is positioned within the distal end of outer coil (302). Navigation coil (310) thus presents an effective outer diameter that is less than the inner diameter defined by outer coil (302) in this example. In addition, the distal portion of navigation coil (310) is positioned within tip member (304). In some versions, a core of iron and/or some other ferromagnetic material is positioned within the inner diameter that is defined by navigation coil (310). Such a core of material may extend along the full length of navigation coil (310) or a portion of the length of navigation coil (310). Navigation coil (310) is configured to cooperate with image guidance system (200) to provide signals indicative of the positioning of the distal end of guidewire (300) within the patient, as described above. Navigation cable (312) is coupled with the proximal end of navigation coil (310) and transmits the signals from navigation coil (310) to image guidance system (200) via cable (210). It should therefore be understood that the proximal end of guidewire (300) may include a connector hub similar to connector hub (152); and that navigation cable (312) may be in communication with the connector hub.

Solder joint (320) is used to secure at least some of the above-described components together. In the present example, solder joint (320) is proximal to tip member (304) and extends about outer coil (302), core wire (308), and navigation coil (310). Navigation cable (312) terminates proximal to the longitudinal position of solder joint (320). In addition to securing components of guidewire (300) together, solder joint (320) may also provide some degree of structural integrity to guidewire (300). It should be understood that solder joint (320) is merely optional such that components of guidewire (300) may be secured together in any other suitable fashion.

B. Exemplary Navigation Guidewire with Coil Sensor Inside Distal Tip Member and Optical Fiber in Coil

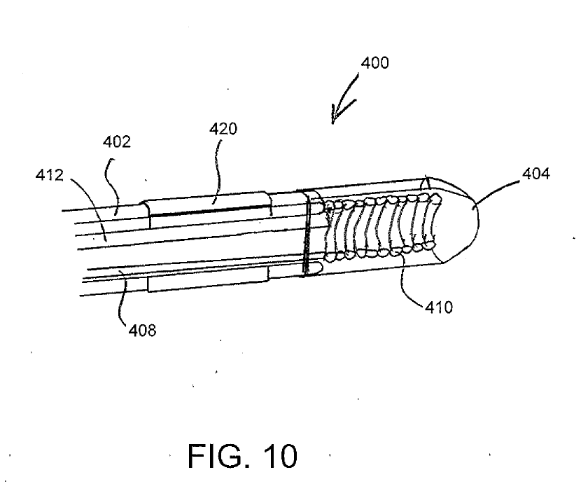

FIG. 10 shows an exemplary guidewire (400) that may be incorporated into dilation catheter system (100) for use with image guidance system (200). Except as otherwise noted herein, guidewire (400) may be constructed and operable just like guidewires (50, 150) described above. Guidewire (400) of this example comprises an outer coil (402), a distal tip member (404), a core wire (408), a navigation coil (410), a navigation cable (412), and a solder joint (420). Outer coil (402) extends along the length of guidewire (400) and contains core wire (408) and navigation cable (412). Outer coil (402) may be constructed in accordance with any suitable conventional guidewire outer coil.

Distal tip member (404) has an atraumatic dome shape and is secured to the distal end of outer coil (402). By way of example only, distal tip member (404) may be formed of an optically transmissive polymeric material and may be secured to the distal end of outer coil (402) using an interference fit, welding, adhesive, or using any other suitable techniques. As another merely illustrative example, distal tip member (404) may be formed by an optically transmissive adhesive that is applied to the distal end of outer coil (402) and then cured. It should also be understood that distal tip member (404) may be configured and operable like lens (58) described above. Other suitable ways in which distal tip member (404) may be configured and operable will be apparent to those of ordinary skill in the art in view of the teachings herein.

In some versions, the distal end of an optical fiber (not shown) is optically coupled with distal tip member (404). The proximal end of the optical fiber is configured to couple with a light source. Various suitable ways in which an optical fiber may be coupled with a light source will be apparent to those of ordinary skill in the art in view of the teachings herein. The optical fiber is configured to provide a path for communication of light from the light source to distal tip member (404), such that distal tip member (404) can emit light generated by the light source. By way of example only one or more optical fibers may run alongside the outer diameter defined by navigation coil (410) in order to reach distal tip member (404). As another merely illustrative example, one or more optical fibers may terminate in the sidewall of outer coil (402) at a location just proximal to navigation coil (410), such that the one or more optical fibers may emit light through the sidewall of outer coil (402). In versions where guidewire (400) includes an optical fiber, it should be understood that any suitable number of optical fibers may be used. Various suitable ways in which guidewire (400) may incorporate one or more optical fibers will be apparent to those of ordinary skill in the art in view of the teachings herein. It should also be understood that guidewire (400) may simply lack any optical fibers.

Core wire (408) is configured to provide additional structural integrity to outer coil (402). In the present example, the proximal end of core wire (408) is fixedly secured to the proximal end of outer coil (402), while the distal end of core wire (408) is fixedly secured to the distal end of outer coil (402). Core wire (408) thus prevents or restricts longitudinal stretching of outer coil (402). Various suitable materials and configurations that may be used to form core wire (408) will be apparent to those of ordinary skill in the art in view of the teachings herein.

Navigation coil (410) is positioned distal to the distal end of outer coil (402). Navigation coil (410) presents an effective outer diameter that is greater than the inner diameter defined by outer coil (402) in this example. In addition, the entire length of navigation coil (410) is positioned within tip member (404). It should be understood that the positioning of navigation coil (410) in this example may enable navigation coil (410) to be formed of a thicker gauge of wire (e.g., in comparison to navigation coil (310)); and that fewer turns of the wire may be needed in order to form navigation coil (410) (e.g., in comparison to navigation coil (310). It should also be understood that positioning navigation coil (410) distal to the distal end of outer coil (402) may present less signal interference than might otherwise be produced in versions such as guidewire (300) where navigation coil (410) is positioned within outer coil (402). In some versions, a core of iron and/or some other ferromagnetic material is positioned within the inner diameter that is defined by navigation coil (410). Such a core of material may extend along the full length of navigation coil (410) or a portion of the length of navigation coil (410).

Navigation coil (410) is configured to cooperate with image guidance system (200) to provide signals indicative of the positioning of the distal end of guidewire (400) within the patient, as described above. Navigation cable (412) is coupled with the proximal end of navigation coil (410) and transmits the signals from navigation coil (410) to image guidance system (200) via cable (210). It should therefore be understood that the proximal end of guidewire (400) may include a connector hub similar to connector hub (152); and that navigation cable (412) may be in communication with the connector hub.

Solder joint (420) is used to secure at least some of the above-described components together. In the present example, solder joint (420) is proximal to tip member (404) and extends about outer coil (402), core wire (408), and a distal portion of navigation cable (412). Navigation coil (410) is located distal to the longitudinal position of solder joint (420). In addition to securing components of guidewire (400) together, solder joint (420) may also provide some degree of structural integrity to guidewire (400). It should be understood that solder joint (420) is merely optional such that components of guidewire (400) may be secured together in any other suitable fashion.

C. Exemplary Navigation Guidewire with Support Tube in Coil Sensor

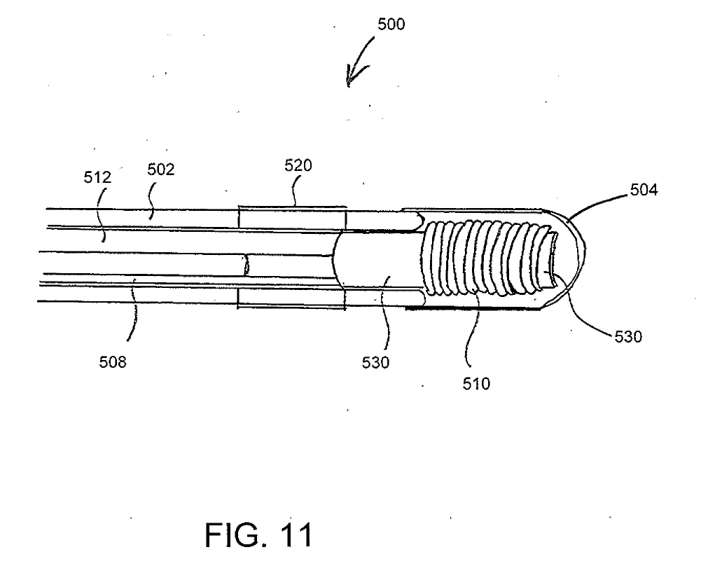

FIG. 11 shows an exemplary guidewire (500) that may be incorporated into dilation catheter system (100) for use with image guidance system (200). Except as otherwise noted herein, guidewire (500) may be constructed and operable just like guidewires (50, 150) described above. Guidewire (500) of this example comprises an outer coil (502), a distal tip member (504), a core wire (508), a navigation coil (510), a navigation cable (512), a solder joint (520), and a support tube (530). Outer coil (502) extends along the length of guidewire (500) and contains core wire (508), navigation cable (512), and a proximal portion of support tube (530). Outer coil (502) may be constructed in accordance with any suitable conventional guidewire outer coil.

Distal tip member (504) has an atraumatic dome shape and is secured to the distal end of outer coil (502). By way of example only, distal tip member (504) may be formed of an optically transmissive polymeric material and may be secured to the distal end of outer coil (502) using an interference fit, welding, adhesive, or using any other suitable techniques. As another merely illustrative example, distal tip member (504) may be formed by an optically transmissive adhesive that is applied to the distal end of outer coil (502) and then cured. It should also be understood that distal tip member (504) may be configured and operable like lens (58) described above. Other suitable ways in which distal tip member (504) may be configured and operable will be apparent to those of ordinary skill in the art in view of the teachings herein.

In the present example, guidewire (500) lacks an optical fiber. It should therefore be understood that some versions of guidewire (500) simply do not emit light through distal tip member (504). Alternatively, the lumen formed by outer coil (502) may form a light pipe that transmits light from the proximal end of outer coil (502) to distal tip member (504). As another merely illustrative alternative, one or more optical fibers may be included in guidewire (500). In such versions, the one or more optical fibers may extend through the interior of support tube (530) to reach distal tip member (504).

Core wire (508) is configured to provide additional structural integrity to outer coil (502). In the present example, the proximal end of core wire (508) is fixedly secured to the proximal end of outer coil (502), while the distal end of core wire (508) is fixedly secured to the distal end of outer coil (502). In some other versions, the distal end of core wire (508) is fixedly secured to support tube (530). In some such versions, support tube (530) is fixedly secured to the distal end of outer coil (502). In any such examples, core wire (508) may prevent or restrict longitudinal stretching of outer coil (502). Various suitable materials and configurations that may be used to form core wire (508) will be apparent to those of ordinary skill in the art in view of the teachings herein.

Navigation coil (510) is positioned distal to the distal end of outer coil (502). Navigation coil (510) presents an effective outer diameter that is greater than the inner diameter defined by outer coil (502) in this example. In addition, the entire length of navigation coil (510) is positioned within tip member (504). It should be understood that the positioning of navigation coil (510) in this example may enable navigation coil (510) to be formed of a thicker gauge of wire (e.g., in comparison to navigation coil (310); and that fewer turns of the wire may be needed in order to form navigation coil (510) (e.g., in comparison to navigation coil (310). It should also be understood that positioning navigation coil (510) distal to the distal end of outer coil (502) may present less signal interference than might otherwise be produced in versions such as guidewire (300) where navigation coil (510) is positioned within outer coil (502). In some versions, a core of iron and/or some other ferromagnetic material is positioned within the inner diameter that is defined by navigation coil (510). Such a core of material may extend along the full length of navigation coil (510) or a portion of the length of navigation coil (510).

Navigation coil (510) is configured to cooperate with image guidance system (200) to provide signals indicative of the positioning of the distal end of guidewire (500) within the patient, as described above. Navigation cable (512) is coupled with the proximal end of navigation coil (510) and transmits the signals from navigation coil (510) to image guidance system (200) via cable (210). It should therefore be understood that the proximal end of guidewire (500) may include a connector hub similar to connector hub (152); and that navigation cable (512) may be in communication with the connector hub.

Solder joint (520) is used to secure at least some of the above-described components together. In the present example, solder joint (520) is proximal to tip member (504) and extends about outer coil (502), core wire (508), a distal portion of navigation cable (512), and a proximal portion of support tube (530). Navigation coil (510) is located distal to the longitudinal position of solder joint (520). In addition to securing components of guidewire (500) together, solder joint (520) may also provide some degree of structural integrity to guidewire (500). It should be understood that solder joint (520) is merely optional such that components of guidewire (500) may be secured together in any other suitable fashion.

Support tube (530) of the present example has a cylindraceous configuration. A proximal portion of support tube (530) is positioned within the distal end of outer coil (502). Support tube (530) thus presents an outer diameter that is less than the inner diameter defined by outer coil (502). Support tube (530) also extends fully through navigation coil (510). In some versions, the distal end of support tube (530) is distal to the distal end of navigation coil (510). In some other versions, the distal end of support tube (530) is flush with the distal end of navigation coil (510). In still other versions, the distal end of support tube (530) is just proximal to the distal end of navigation coil (510). It should also be understood that the outer diameter of support tube (530) may have any suitable relationship with the effective inner diameter defined by navigation coil (510). In some versions, navigation coil (510) is wrapped about the exterior of support tube (530) such that the interior of navigation coil (510) contacts the exterior of support tube (530). In some other versions, the interior of navigation coil (510) is spaced radially outwardly from the exterior of support tube (530). It should also be understood that support tube (530) may include a transversely oriented opening or slot, etc., providing a pathway for navigation cable (512) to couple with navigation coil (510).

Support tube (530) of the present example provides further structural integrity to navigation coil (510) (e.g., as compared to navigation coil (310, 410)), reducing the likelihood that navigation coil (510) will be damaged as tip member (504) bumps into anatomical structures within the patient and other structures during use of guidewire (500). Support tube (530) of the present example is also configured to not have an adverse impact on the signal provided by navigation coil (510). In some versions, support tube (530) is constructed of a non-conductive polymeric material. Other suitable ways in which support tube (530) may be configured will be apparent to those of ordinary skill in the art in view of the teachings herein.

D. Exemplary Navigation Guidewire with Support Tube about Coil Sensor

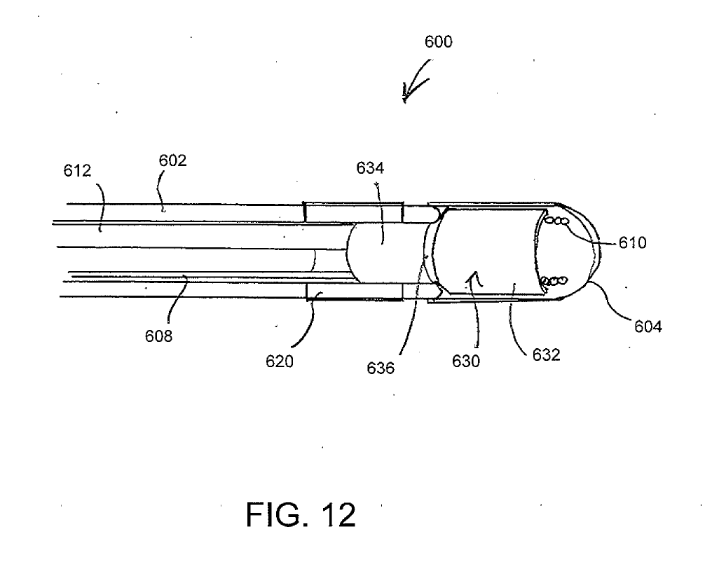

FIG. 12 shows an exemplary guidewire (600) that may be incorporated into dilation catheter system (100) for use with image guidance system (200). Except as otherwise noted herein, guidewire (600) may be constructed and operable just like guidewires (50, 150) described above. Guidewire (600) of this example comprises an outer coil (602), a distal tip member (604), a core wire (608), a navigation coil (610), a navigation cable (612), a solder joint (620), and a support tube (630). Outer coil (602) extends along the length of guidewire (600) and contains core wire (608), navigation cable (612), and a proximal portion (634) of support tube (630). Outer coil (602) may be constructed in accordance with any suitable conventional guidewire outer coil.

Distal tip member (604) has an atraumatic dome shape and is secured to the distal end of outer coil (602). By way of example only, distal tip member (604) may be formed of an optically transmissive polymeric material and may be secured to the distal end of outer coil (602) using an interference fit, welding, adhesive, or using any other suitable techniques. As another merely illustrative example, distal tip member (604) may be formed by an optically transmissive adhesive that is applied to the distal end of outer coil (602) and then cured. It should also be understood that distal tip member (604) may be configured and operable like lens (58) described above. Other suitable ways in which distal tip member (604) may be configured and operable will be apparent to those of ordinary skill in the art in view of the teachings herein.

In the present example, guidewire (600) lacks an optical fiber. It should therefore be understood that some versions of guidewire (600) simply do not emit light through distal tip member (604). Alternatively, the lumen formed by outer coil (602) may form a light pipe that transmits light from the proximal end of outer coil (602) to distal tip member (604). As another merely illustrative alternative, one or more optical fibers may be included in guidewire (600). In such versions, the one or more optical fibers may extend through the interior of support tube (630) to reach distal tip member (604).

Core wire (608) is configured to provide additional structural integrity to outer coil (602). In the present example, the proximal end of core wire (608) is fixedly secured to the proximal end of outer coil (602), while the distal end of core wire (608) is fixedly secured to the distal end of outer coil (602). In some other versions, the distal end of core wire (608) is fixedly secured to support tube (630). In some such versions, support tube (630) is fixedly secured to the distal end of outer coil (602). In any such examples, core wire (608) may prevent or restrict longitudinal stretching of outer coil (602). Various suitable materials and configurations that may be used to form core wire (608) will be apparent to those of ordinary skill in the art in view of the teachings herein.

Navigation coil (610) is positioned distal to the distal end of outer coil (602). Navigation coil (610) presents an effective outer diameter that is greater than the inner diameter defined by outer coil (602) in this example. In addition, the entire length of navigation coil (610) is positioned within tip member (604). It should be understood that the positioning of navigation coil (610) in this example may enable navigation coil (610) to be formed of a thicker gauge of wire (e.g., in comparison to navigation coil (310); and that fewer turns of the wire may be needed in order to form navigation coil (610) (e.g., in comparison to navigation coil (310). It should also be understood that positioning navigation coil (610) distal to the distal end of outer coil (602) may present less signal interference than might otherwise be produced in versions such as guidewire (300) where navigation coil (610) is positioned within outer coil (602). In some versions, a core of iron and/or some other ferromagnetic material is positioned within the inner diameter that is defined by navigation coil (610). Such a core of material may extend along the full length of navigation coil (610) or a portion of the length of navigation coil (610).

Navigation coil (610) is configured to cooperate with image guidance system (200) to provide signals indicative of the positioning of the distal end of guidewire (600) within the patient, as described above. Navigation cable (612) is coupled with the proximal end of navigation coil (610) and transmits the signals from navigation coil (610) to image guidance system (200) via cable (210). It should therefore be understood that the proximal end of guidewire (600) may include a connector hub similar to connector hub (152); and that navigation cable (612) may be in communication with the connector hub.

Solder joint (620) is used to secure at least some of the above-described components together. In the present example, solder joint (620) is proximal to tip member (604) and extends about outer coil (602), core wire (608), a distal portion of navigation cable (612), and a proximal portion (634) of support tube (630). Navigation coil (610) is located distal to the longitudinal position of solder joint (620). In addition to securing components of guidewire (600) together, solder joint (620) may also provide some degree of structural integrity to guidewire (600). It should be understood that solder joint (620) is merely optional such that components of guidewire (600) may be secured together in any other suitable fashion.

Support tube (630) of the present example has a distal portion (632), a proximal portion (634), and a transition portion (636). Distal portion (632) has an outer diameter and an inner diameter that are greater than the outer diameter and inner diameter, respectively, of proximal portion (634). Transition portion (636) provides a tapered transition between portions (632, 634) in this example, though it should be understood that the transition may alternatively be stepped or otherwise configured. Proximal portion (634) is positioned within the distal end of outer coil (602). Proximal portion (634) thus presents an outer diameter that is less than the inner diameter defined by outer coil (602). Distal portion (632) is positioned distal to the distal end of outer coil (602) and has an outer diameter that is greater than the inner diameter defined by outer coil (602). Transition portion (636) is positioned right at the distal end of outer coil (602).

Distal portion (632) extends about navigation coil (610). Distal portion (632) thus has an inner diameter that is greater than the effective outer diameter presented by navigation coil (610). In some versions, the distal end of support tube (630) is distal to the distal end of navigation coil (610). In some other versions, the distal end of support tube (630) is flush with the distal end of navigation coil (610). In still other versions, the distal end of support tube (630) is just proximal to the distal end of navigation coil (610). It should also be understood that the inner diameter of distal portion (632) may have any suitable relationship with the effective outer diameter defined by navigation coil (610). In some versions, the exterior of navigation coil (610) contacts the interior of distal portion (632). In some other versions, the exterior of navigation coil (610) is spaced radially inwardly from the interior of distal portion (632). It should also be understood that navigation cable (612) may couple with navigation coil (610) somewhere within distal portion (632).

Support tube (630) of the present example provides further structural integrity to navigation coil (610) (e.g., as compared to navigation coil (410)), reducing the likelihood that navigation coil (610) will be damaged as tip member (604) bumps into anatomical structures within the patient and other structures during use of guidewire (600). Support tube (630) of the present example is also configured to not have an adverse impact on the signal provided by navigation coil (610). In some versions, support tube (630) is constructed of a non-conductive polymeric material. Other suitable ways in which support tube (630) may be configured will be apparent to those of ordinary skill in the art in view of the teachings herein.

E. Exemplary Navigation Guidewire with Support Tube about Coil Sensor and Core Wire in Coil Sensor

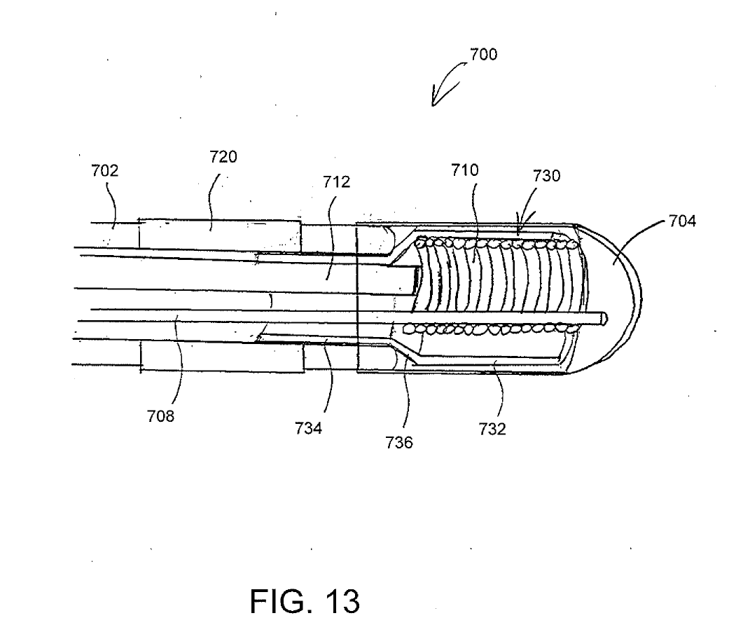

FIG. 13 shows an exemplary guidewire (700) that may be incorporated into dilation catheter system (100) for use with image guidance system (200). Except as otherwise noted herein, guidewire (700) may be constructed and operable just like guidewires (50, 150) described above. Guidewire (700) of this example comprises an outer coil (702), a distal tip member (704), a core wire (708), a navigation coil (710), a navigation cable (712), a solder joint (720), and a support tube (730). Outer coil (702) extends along the length of guidewire (700) and contains core wire (708), navigation cable (712), and a proximal portion (734) of support tube (730). Outer coil (702) may be constructed in accordance with any suitable conventional guidewire outer coil.

Distal tip member (704) has an atraumatic dome shape and is secured to the distal end of outer coil (702). By way of example only, distal tip member (704) may be formed of an optically transmissive polymeric material and may be secured to the distal end of outer coil (702) using an interference fit, welding, adhesive, or using any other suitable techniques. As another merely illustrative example, distal tip member (704) may be formed by an optically transmissive adhesive that is applied to the distal end of outer coil (702) and then cured. It should also be understood that distal tip member (704) may be configured and operable like lens (58) described above. Other suitable ways in which distal tip member (704) may be configured and operable will be apparent to those of ordinary skill in the art in view of the teachings herein.

In the present example, guidewire (700) lacks an optical fiber. It should therefore be understood that some versions of guidewire (700) simply do not emit light through distal tip member (704). Alternatively, the lumen formed by outer coil (702) may form a light pipe that transmits light from the proximal end of outer coil (702) to distal tip member (704). As another merely illustrative alternative, one or more optical fibers may be included in guidewire (700). In such versions, the one or more optical fibers may extend through the interior of support tube (730) to reach distal tip member (704).

Core wire (708) is configured to provide additional structural integrity to outer coil (702). In the present example, the proximal end of core wire (708) is fixedly secured to the proximal end of outer coil (702), while the distal end of core wire (708) extends all the way into distal tip member (704). The distal end of core wire (708) is positioned distal to the distal end of navigation coil (710) in this example. Core wire (708) may prevent or restrict longitudinal stretching of outer coil (702). Various suitable materials and configurations that may be used to form core wire (708) will be apparent to those of ordinary skill in the art in view of the teachings herein.

Navigation coil (710) is positioned distal to the distal end of outer coil (702). Navigation coil (710) presents an effective outer diameter that is greater than the inner diameter defined by outer coil (702) in this example. In addition, the entire length of navigation coil (710) is positioned within tip member (704). It should be understood that the positioning of navigation coil (710) in this example may enable navigation coil (710) to be formed of a thicker gauge of wire (e.g., in comparison to navigation coil (310); and that fewer turns of the wire may be needed in order to form navigation coil (710) (e.g., in comparison to navigation coil (310). It should also be understood that positioning navigation coil (710) distal to the distal end of outer coil (702) may present less signal interference than might otherwise be produced in versions such as guidewire (300) where navigation coil (710) is positioned within outer coil (702). In some versions, a core of iron and/or some other ferromagnetic material is positioned within the inner diameter that is defined by navigation coil (710). Such a core of material may extend along the full length of navigation coil (710) or a portion of the length of navigation coil (710).

Navigation coil (710) is configured to cooperate with image guidance system (200) to provide signals indicative of the positioning of the distal end of guidewire (700) within the patient, as described above. Navigation cable (712) is coupled with the proximal end of navigation coil (710) and transmits the signals from navigation coil (710) to image guidance system (200) via cable (210). It should therefore be understood that the proximal end of guidewire (700) may include a connector hub similar to connector hub (152); and that navigation cable (712) may be in communication with the connector hub.

Solder joint (720) is used to secure at least some of the above-described components together. In the present example, solder joint (720) is proximal to tip member (704) and extends about outer coil (702), core wire (708), a distal portion of navigation cable (712), and a proximal portion (734) of support tube (730). Navigation coil (710) is located distal to the longitudinal position of solder joint (720). In addition to securing components of guidewire (700) together, solder joint (720) may also provide some degree of structural integrity to guidewire (700). It should be understood that solder joint (720) is merely optional such that components of guidewire (700) may be secured together in any other suitable fashion. In some exemplary alternative versions, solder joint (720) is positioned proximal to proximal portion (734) of support tube (732). In some such versions, a length of outer coil (702) between the distal end of solder joint (720) and the proximal end of proximal portion (734) may act as a shock absorber, providing deflection to absorb impacts when distal tip member (704) bumps into anatomical structures or other structures during use of guidewire (700).

Support tube (730) of the present example has a distal portion (732), a proximal portion (734), and a transition portion (736). Distal portion (732) has an outer diameter and an inner diameter that are greater than the outer diameter and inner diameter, respectively, of proximal portion (734). Transition portion (736) provides a tapered transition between portions (732, 734) in this example, though it should be understood that the transition may alternatively be stepped or otherwise configured. Proximal portion (734) is positioned within the distal end of outer coil (702). Proximal portion (734) thus presents an outer diameter that is less than the inner diameter defined by outer coil (702). Distal portion (732) is positioned distal to the distal end of outer coil (702) and has an outer diameter that is greater than the inner diameter defined by outer coil (702). Transition portion (736) is positioned right at the distal end of outer coil (702).

Distal portion (732) extends about navigation coil (710). Distal portion (732) thus has an inner diameter that is greater than the effective outer diameter presented by navigation coil (710). In some versions, the distal end of support tube (730) is distal to the distal end of navigation coil (710). In some other versions, the distal end of support tube (730) is flush with the distal end of navigation coil (710). In still other versions, the distal end of support tube (730) is just proximal to the distal end of navigation coil (710). It should also be understood that the inner diameter of distal portion (732) may have any suitable relationship with the effective outer diameter defined by navigation coil (710). In some versions, the exterior of navigation coil (710) contacts the interior of distal portion (732). In some other versions, the exterior of navigation coil (710) is spaced radially inwardly from the interior of distal portion (732). It should also be understood that navigation cable (712) may couple with navigation coil (710) somewhere within distal portion (732).

Support tube (730) of the present example provides further structural integrity to navigation coil (710) (e.g., as compared to navigation coil (410)), reducing the likelihood that navigation coil (710) will be damaged as tip member (704) bumps into anatomical structures within the patient and other structures during use of guidewire (700). It should be understood that the extension of core wire (708) through the interior of navigation coil (710), as well as the fixation of the distal end of core wire (708) within distal tip member (704) at a location distal to the distal end of navigation coil (710), may also further enhance the structural integrity of navigation coil (710). This distal extension of core wire (708) may be provided in any of the other examples described herein as well. Support tube (730) of the present example is also configured to not have an adverse impact on the signal provided by navigation coil (710). In some versions, support tube (730) is constructed of a non-conductive polymeric material. Other suitable ways in which support tube (730) may be configured will be apparent to those of ordinary skill in the art in view of the teachings herein.

F. Exemplary Navigation Guidewire with Coil Sensor within Extrusion

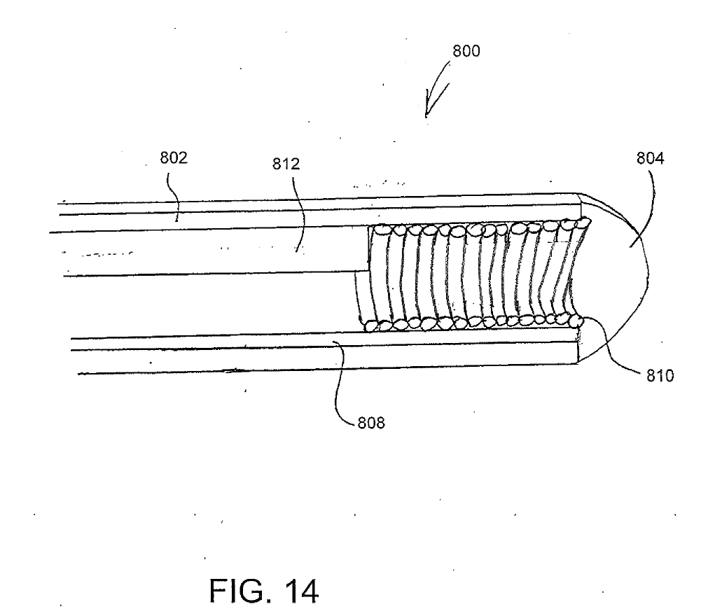

FIG. 14 shows an exemplary guidewire (800) that may be incorporated into dilation catheter system (100) for use with image guidance system (200). Except as otherwise noted herein, guidewire (800) may be constructed and operable just like guidewires (50, 150) described above. Guidewire (800) of this example comprises an outer extrusion (802), a distal tip member (804), a core wire (808), a navigation coil (810), and a navigation cable (812). Outer extrusion (802) extends along the length of guidewire (800) and contains core wire (808), a proximal portion of navigation coil (810), and navigation cable (812).

In the present example, outer extrusion (802) comprises a single unitary extrusion of a polymeric material. By way of example only, the material may comprise polyether block amide (PEBA) such as PEBAX® (by Arkema Inc. of King of Prussia, Pa.); nylon; kevlar; and/or any other suitable material(s). It should also be understood that the polymer material may be provided in a braided form in addition to or in lieu of being provided in an extruded form. It should be understood that outer extrusion (802) is used instead of an outer coil as is used in various other examples described herein. Outer extrusion (802) may have a thinner wall thickness than the effective wall thickness that would otherwise be provided by an outer coil as used in other examples described herein. This may enable outer extrusion (802) to define a greater inner diameter than would otherwise be defined by an outer coil as used in other examples described herein. In addition, outer extrusion (802) may provide less signal interference with navigation coil (810) than might otherwise be provided by outer coils as used in other examples herein. Other than the differences outlined above (among other potential differences), outer extrusion (802) may otherwise perform similar to outer coils as used in other examples described herein.

Distal tip member (804) has an atraumatic dome shape and is secured to the distal end of outer extrusion (802). By way of example only, distal tip member (804) may be formed of an optically transmissive polymeric material and may be secured to the distal end of outer extrusion (802) using an interference fit, welding, adhesive, or using any other suitable techniques. As another merely illustrative example, distal tip member (804) may be formed by an optically transmissive adhesive that is applied to the distal end of outer extrusion (802) and then cured. It should also be understood that distal tip member (804) may be configured and operable like lens (58) described above. Other suitable ways in which distal tip member (804) may be configured and operable will be apparent to those of ordinary skill in the art in view of the teachings herein.

In some versions, the distal end of an optical fiber (not shown) is optically coupled with distal tip member (804). The proximal end of the optical fiber is configured to couple with a light source. Various suitable ways in which an optical fiber may be coupled with a light source will be apparent to those of ordinary skill in the art in view of the teachings herein. The optical fiber is configured to provide a path for communication of light from the light source to distal tip member (804), such that distal tip member (804) can emit light generated by the light source. By way of example only one or more optical fibers may run alongside the outer diameter defined by navigation coil (810) in order to reach distal tip member (804). As another merely illustrative example, one or more optical fibers may terminate in the sidewall of outer extrusion (802) at a location just proximal to navigation coil (810), such that the one or more optical fibers may emit light through the sidewall of outer extrusion (802). In versions where guidewire (800) includes an optical fiber, it should be understood that any suitable number of optical fibers may be used. Various suitable ways in which guidewire (800) may incorporate one or more optical fibers will be apparent to those of ordinary skill in the art in view of the teachings herein. It should also be understood that guidewire (800) may simply lack any optical fibers.

Core wire (808) is configured to provide additional structural integrity to outer extrusion (802). In the present example, the proximal end of core wire (808) is fixedly secured to the proximal end of outer extrusion (802), while the distal end of core wire (808) is fixedly secured to the distal end of outer extrusion (802). Core wire (808) thus prevents or restricts longitudinal stretching of outer extrusion (802). Various suitable materials and configurations that may be used to form core wire (808) will be apparent to those of ordinary skill in the art in view of the teachings herein. While core wire (808) is shown as being positioned outside of navigation coil (810) in this example, it should be understood that core wire (808) may be positioned inside of navigation coil (810) in other examples.

Navigation coil (810) is positioned within the distal end of outer extrusion (802). Navigation coil (810) thus presents an effective outer diameter that is less than the inner diameter defined by outer extrusion (802) in this example. In addition, the distal portion of navigation coil (810) is positioned within tip member (804). With outer extrusion (802) having an inner diameter that is larger than the inner diameter defined by outer coil (302), the larger inner diameter may enable navigation coil (810) to be formed of a thicker gauge of wire (e.g., in comparison to navigation coil (310)); and that fewer turns of the wire may be needed in order to form navigation coil (810) (e.g., in comparison to navigation coil (310). In some versions, a core of iron and/or some other ferromagnetic material is positioned within the inner diameter that is defined by navigation coil (810). Such a core of material may extend along the full length of navigation coil (810) or a portion of the length of navigation coil (810).

Navigation coil (810) is configured to cooperate with image guidance system (200) to provide signals indicative of the positioning of the distal end of guidewire (800) within the patient, as described above. Navigation cable (812) is coupled with the proximal end of navigation coil (810) and transmits the signals from navigation coil (810) to image guidance system (200) via cable (210). It should therefore be understood that the proximal end of guidewire (800) may include a connector hub similar to connector hub (152); and that navigation cable (812) may be in communication with the connector hub.

Guidewire (800) of the present example lacks a solder joint due to the construction of outer extrusion (802). It should be understood, however, that one or more reinforcement sleeves of any suitable length(s) may be positioned at any suitable location(s) along the length of guidewire (800). Other modifications that may be made to guidewire (800) will be apparent to those of ordinary skill in the art in view of the teachings herein.

G. Exemplary Navigation Guidewire with Core in Coil Sensor

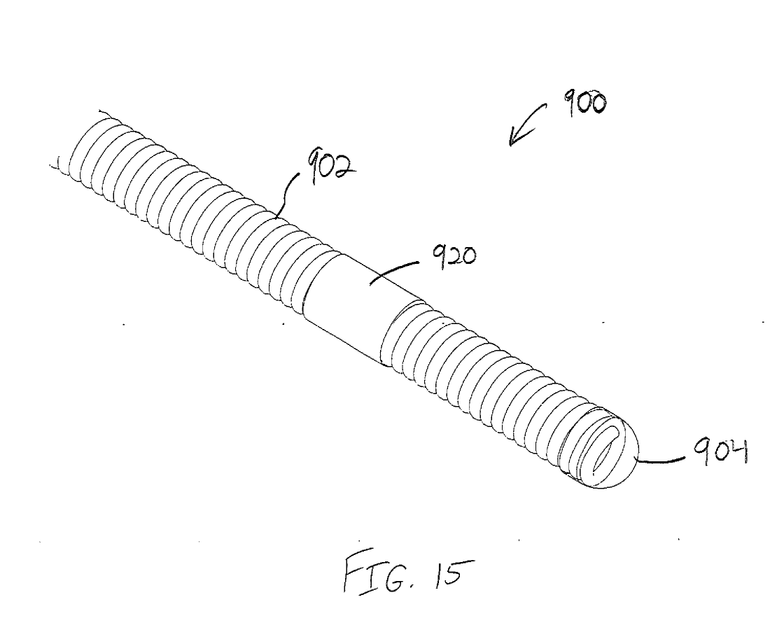

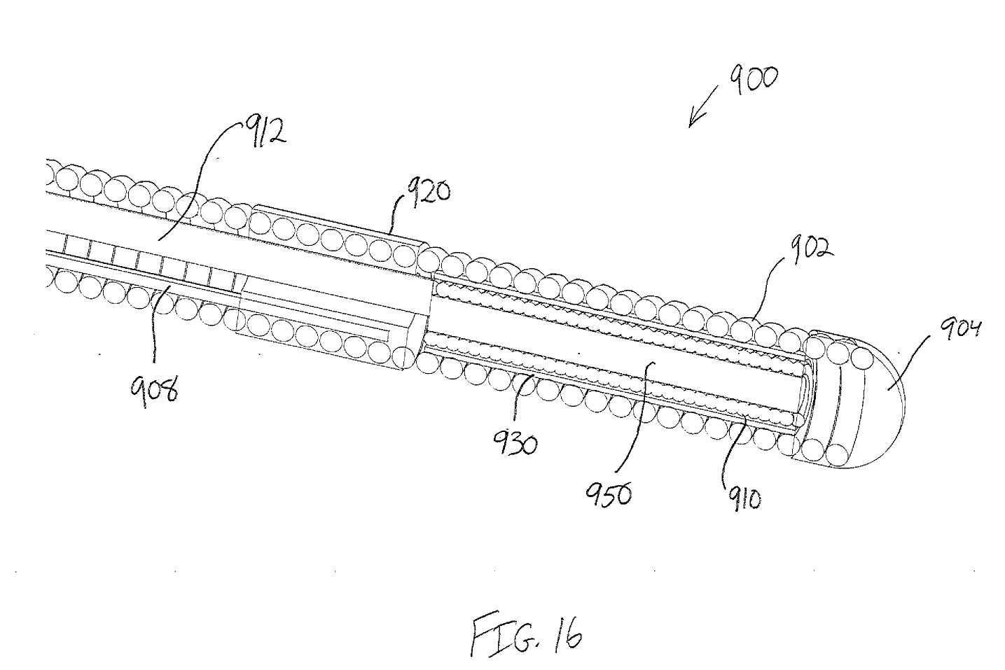

FIGS. 15-16 show another exemplary guidewire (900) that may be incorporated into dilation catheter system (100) for use with image guidance system (200). Except as otherwise noted herein, guidewire (900) may be constructed and operable just like guidewires (50, 150) described above. Guidewire (900) of this example comprises an outer coil (902), a distal tip member (904), a core wire (908), a navigation coil (910), a navigation cable (912), and a solder joint (920). Outer coil (902) extends along the length of guidewire (900) and contains core wire (908), a proximal portion of navigation coil (910), and navigation cable (912). Outer coil (902) may be constructed in accordance with any suitable conventional guidewire outer coil.

Distal tip member (904) has an atraumatic dome shape and is secured to the distal end of outer coil (902). By way of example only, distal tip member (904) may be formed of an optically transmissive polymeric material and may be secured to the distal end of outer coil (902) using an interference fit, welding, adhesive, or using any other suitable techniques. As another merely illustrative example, distal tip member (904) may be formed by an optically transmissive adhesive that is applied to the distal end of outer coil (902) and then cured. It should also be understood that distal tip member (904) may be configured and operable like lens (58) described above.

In addition or in the alternative to being configured like lens (58), distal tip member (904) may comprise an electrically conductive material (e.g., gold or silver filled epoxy, etc.). As another merely illustrative example, distal tip member (904) may comprise a cap that is formed of an electrically conductive metal and/or some other electrically conductive material. Such a cap may be press-fit into the distal end of outer coil (902) and/or soldered to the distal end of outer coil (902). In versions where distal tip member (904) includes an electrically conductive material, the conductive material may be selected such that it has a relatively low magnetic permeability while having good electrical conductivity. Also in versions where distal tip member (904) includes an electrically conductive material, distal tip member may be in electrical continuity with outer coil (902), which may also be formed of an electrically conductive material. In the present example, outer coil (902) is grounded. Thus, the combination of an electrically conductive distal tip member (904) and outer coil (902) creates an electrical shield (e.g., similar to a Faraday cage), though it is transparent to the magnetic field. The combination may thus reduce electrical coupling to guidewire (900) such as capacitive/faradic coupling caused by the distal end of guidewire (900) coming into contact with the patient's body. Other suitable ways in which distal tip member (904) may be configured and operable will be apparent to those of ordinary skill in the art in view of the teachings herein.

In some versions, the distal end of an optical fiber (not shown) is optically coupled with distal tip member (904). The proximal end of the optical fiber is configured to couple with a light source. Various suitable ways in which an optical fiber may be coupled with a light source will be apparent to those of ordinary skill in the art in view of the teachings herein. The optical fiber is configured to provide a path for communication of light from the light source to distal tip member (904), such that distal tip member (904) can emit light generated by the light source. By way of example only one or more optical fibers may run alongside the outer diameter defined by navigation coil (910) in order to reach distal tip member (904). As another merely illustrative example, one or more optical fibers may terminate in the sidewall of outer coil (902) at a location just proximal to navigation coil (910), such that the one or more optical fibers may emit light through the sidewall of outer coil (902). In versions where guidewire (900) includes an optical fiber, it should be understood that any suitable number of optical fibers may be used. Various suitable ways in which guidewire (900) may incorporate one or more optical fibers will be apparent to those of ordinary skill in the art in view of the teachings herein. It should also be understood that guidewire (900) may simply lack any optical fibers.

Core wire (908) is configured to provide additional structural integrity to outer coil (902). In the present example, the proximal end of core wire (908) is fixedly secured to the proximal end of outer coil (902), while the distal end of core wire (908) is fixedly secured to the distal end of outer coil (902). Core wire (908) thus prevents or restricts longitudinal stretching of outer coil (902). Various suitable materials and configurations that may be used to form core wire (908) will be apparent to those of ordinary skill in the art in view of the teachings herein.

Navigation coil (910) is positioned within the distal end of outer coil (902). Navigation coil (910) thus presents an effective outer diameter that is less than the inner diameter defined by outer coil (902) in this example. In addition, the distal end of navigation coil (910) is positioned just proximal to the proximal face of tip member (904). In the present example, a core (950) of ferromagnetic material is positioned within the inner diameter that is defined by navigation coil (910). Core (950) extends along the full length of navigation coil (910) in this example. By way of example only, core (950) may be formed of iron or some other ferromagnetic material. Navigation coil (910) is configured to cooperate with image guidance system (200) to provide signals indicative of the positioning of the distal end of guidewire (900) within the patient, as described above. Navigation cable (912) is coupled with the proximal end of navigation coil (910) and transmits the signals from navigation coil (910) to image guidance system (200) via cable (210). It should therefore be understood that the proximal end of guidewire (900) may include a connector hub similar to connector hub (152); and that navigation cable (912) may be in communication with the connector hub.

A support tube (930) is positioned about navigation coil (910) in the present example. Support tube (930) of the present example has a cylindraceous configuration. Support tube (930) thus presents an outer diameter that is less than the inner diameter defined by outer coil (902). Support tube (930) extends along the full length of navigation coil (910), such that the distal and proximal ends of support tube (930) are flush with the distal end proximal ends of navigation coil (910). Alternatively, support tube (930) may have any other suitable length and/or positioning in relation to the length and/or positioning of navigation coil (910). In the present example, the outer surface of support tube (930) is adhered to the inner surface of outer coil (902) by an adhesive; and the inner surface of support tube (930) is adhered to the outer surface of navigation coil (910) by adhesive. Alternatively, any other suitable methods may be used to secure support tube (930) to outer coil (902) and/or navigation coil (910). It should also be understood that support tube (930) may alternatively be secured to just one coil (902, 910) without also being secured to the other coil (902, 910).

Support tube (930) of the present example provides further structural integrity to navigation coil (910) (e.g., as compared to navigation coil (310, 410, 810)), reducing the likelihood that navigation coil (910) will be damaged as tip member (904) bumps into anatomical structures within the patient and other structures during use of guidewire (900). Support tube (930) of the present example is also configured to not have an adverse impact on the signal provided by navigation coil (910). In some versions, support tube (930) is constructed of a non-conductive polymeric material such as polyamide. Other suitable ways in which support tube (930) may be configured will be apparent to those of ordinary skill in the art in view of the teachings herein.

Solder joint (920) is used to secure at least some of the above-described components together. In the present example, solder joint (920) is proximal to tip member (904) and extends about outer coil (902), core wire (908), and navigation cable (912). Navigation coil (910) proximally terminates distal to the longitudinal position of solder joint (920). In addition to securing components of guidewire (900) together, solder joint (920) may also provide some degree of structural integrity to guidewire (900). It should be understood that solder joint (920) is merely optional such that components of guidewire (900) may be secured together in any other suitable fashion.

By way of example only, outer coil (902) may have an effective outer diameter of approximately 0.035 inches and an inner diameter of approximately 0.022 inches. Outer coil (902) may also be formed by a 316 stainless steel (or nitinol) wire having a thickness of approximately 0.006 inches, with a round cross-sectional profile. Navigation coil (910) may have a length of approximately 0.118 inches and an effective outer diameter of approximately 0.022 inches. Core (950) may have an outer diameter of approximately 0.010 inches. Of course, all of these dimensions are just merely illustrative examples. Other suitable dimensions will be apparent to those of ordinary skill in the art in view of the teachings herein.

H. Exemplary Navigation Guidewire with Stacked Outer Coil and Coil Sensor

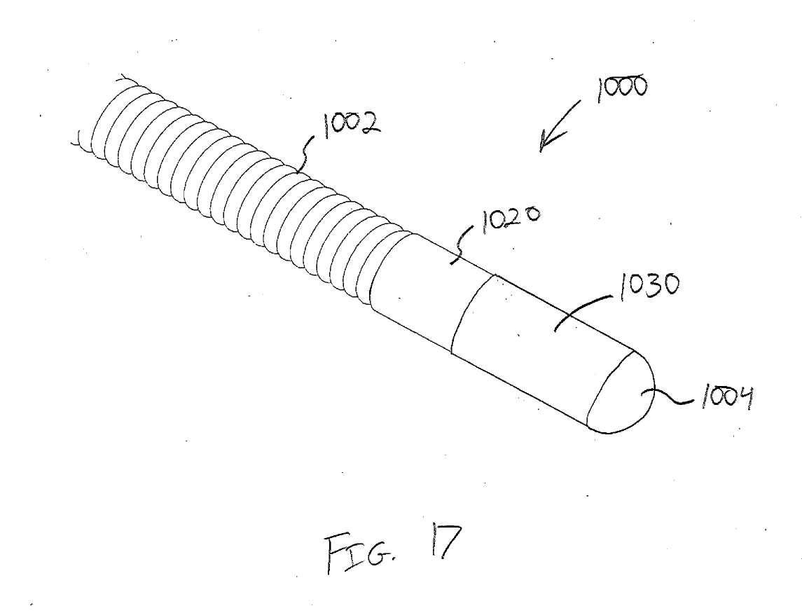

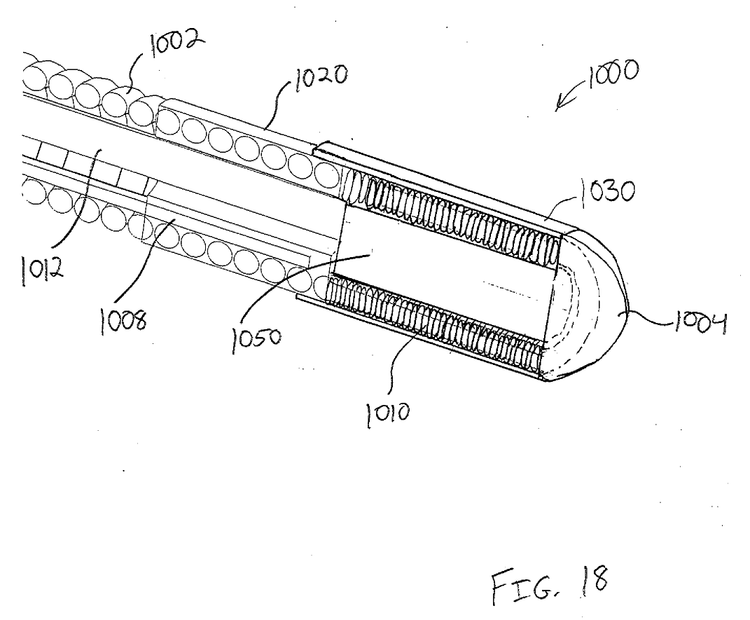

FIGS. 17-18 show another exemplary guidewire (1000) that may be incorporated into dilation catheter system (100) for use with image guidance system (200). Except as otherwise noted herein, guidewire (1000) may be constructed and operable just like guidewires (50, 150) described above. Guidewire (1000) of this example comprises an outer coil (1002), a distal tip member (1004), a core wire (1008), a navigation coil (1010), a navigation cable (1012), a solder joint (1020), and an outer tube (1030) surrounding navigation coil (1010).

Outer coil (1002) extends along a substantial portion of the length of guidewire (900) and contains core wire (1008) and navigation cable (1012). Outer coil (1002) may be constructed in accordance with any suitable conventional guidewire outer coil. Unlike other examples described herein, outer coil (1002) distally terminates at solder joint (1020), which is located the proximal end of navigation coil (1010) and at the proximal end of outer tube (1030). In some versions, outer coil (1002) is formed by a round wire that is wrapped in a helical configuration. In some other versions, outer coil (1002) is formed by a flat wire that is wrapped in a helical configuration. Other suitable ways in which outer coil (1002) may be formed will be apparent to those of ordinary skill in the art in view of the teachings herein.

Distal tip member (1004) has an atraumatic dome shape and is secured to the distal end of outer tube (1030), at the distal end of navigation coil (1010). By way of example only, distal tip member (1004) may be formed of an optically transmissive polymeric material and may be secured to the distal end of outer tube (1030) using an interference fit, welding, adhesive, or using any other suitable techniques. As another merely illustrative example, distal tip member (1004) may be formed by an optically transmissive adhesive that is applied to the distal end of outer tube (1030) and then cured. It should also be understood that distal tip member (1004) may be configured and operable like lens (58) described above. In some variations, however, distal tip member (1004) is not optically transmissive at all. Other suitable ways in which distal tip member (1004) may be configured and operable will be apparent to those of ordinary skill in the art in view of the teachings herein.

In some versions (e.g., versions where distal tip member (1004) is optically transmissive), the distal end of an optical fiber (not shown) is optically coupled with distal tip member (1004). The proximal end of the optical fiber is configured to couple with a light source. Various suitable ways in which an optical fiber may be coupled with a light source will be apparent to those of ordinary skill in the art in view of the teachings herein. The optical fiber is configured to provide a path for communication of light from the light source to distal tip member (1004), such that distal tip member (1004) can emit light generated by the light source. By way of example only one or more optical fibers may run alongside the outer diameter defined by navigation coil (1010) in order to reach distal tip member (1004). As another merely illustrative example, one or more optical fibers may terminate in the sidewall of outer coil (1002) at a location just proximal outer tube (1030), such that the one or more optical fibers may emit light through the sidewall of outer tube (1030). In versions where guidewire (1000) includes an optical fiber, it should be understood that any suitable number of optical fibers may be used. Various suitable ways in which guidewire (1000) may incorporate one or more optical fibers will be apparent to those of ordinary skill in the art in view of the teachings herein. It should also be understood that guidewire (1000) may simply lack any optical fibers.

Core wire (1008) is configured to provide additional structural integrity to outer coil (1002). In the present example, the proximal end of core wire (1008) is fixedly secured to the proximal end of outer coil (1002), while the distal end of core wire (1008) is fixedly secured to the distal end of outer coil (1002). Core wire (1008) thus prevents or restricts longitudinal stretching of outer coil (1002). Various suitable materials and configurations that may be used to form core wire (1008) will be apparent to those of ordinary skill in the art in view of the teachings herein.

Navigation coil (1010) is positioned distal the distal end of outer coil (1002) and within outer tube (1030), such that coils (1002, 1010) are in a longitudinally stacked relationship. In the present example, navigation coil (1010) presents an effective outer diameter that is greater than the inner diameter defined by outer coil (1002) in this example. In some versions, the configuration of guidewire (1000) allows navigation coil (1010) to have an effective outer diameter that is larger than the effective outer diameter of navigation coils in other guidewires described herein. This may provide navigation coil (1010) with a greater sensitivity to fields generated by image guidance system (200), thereby making guidewire (1000) more useful in navigation than other guidewires described herein. In addition or in the alternative, the configuration of guidewire (1000) allows outer coil (1002) to have an effective outer diameter that is smaller than the effective outer diameter of outer coils in other guidewires described herein. It should also be understood that the configuration of guidewire (1000) allows navigation coil (1010) to have an effective length that is shorter than the effective length of navigation coils in other guidewires described herein. This reduction in effective length may effectively reduce the length of the relatively stiff section at the distal end of guidewire (1000), as compared to other guidewire construction described herein. By having a shorter stiff section at the distal end of guidewire (1000), guidewire (1000) may be capable of accessing a greater variety of anatomical structures.

The distal end of navigation coil (1010) is positioned just proximal to the proximal face of tip member (1004). In the present example, a core (1050) of ferromagnetic material is positioned within the inner diameter that is defined by navigation coil (1010). Core (1050) extends along the full length of navigation coil (1010) in this example. By way of example only, core (1050) may be formed of iron or some other ferromagnetic material. Navigation coil (1010) is configured to cooperate with image guidance system (200) to provide signals indicative of the positioning of the distal end of guidewire (1000) within the patient, as described above. Navigation cable (1012) is coupled with the proximal end of navigation coil (1010) and transmits the signals from navigation coil (1010) to image guidance system (200) via cable (210). It should therefore be understood that the proximal end of guidewire (1000) may include a connector hub similar to connector hub (152); and that navigation cable (1012) may be in communication with the connector hub.

As noted above, outer tube (1030) is positioned about navigation coil (1010) in the present example and extends from the distal end of outer coil (1002) to the proximal end of tip member (1004). Outer tube (1030) of the present example has a cylindraceous configuration. Outer tube (1030) presents an inner diameter that is larger than the outer diameter defined by outer coil (1002), such that the distal end of outer coil (1002) fits within the proximal end of outer tube (1030). Outer tube (1030) extends beyond the full length of navigation coil (1010). In the present example, navigation coil (1010) is adhered to the inner surface of outer tube (1030) by an adhesive. Outer tube (1030) is also secured to outer coil (1002) and/or solder joint (1020) by an adhesive. As another merely illustrative example, outer tube (1030) may be secured to the distal end of outer coil (1002) through a lap joint. Alternatively, any other suitable methods may be used to secure outer tube (1030) to outer coil (1002) and/or navigation coil (1010).

Outer tube (1030) of the present example provides further structural integrity to navigation coil (1010) (e.g., as compared to navigation coil (310, 410, 810)), reducing the likelihood that navigation coil (1010) will be damaged as tip member (1004) bumps into anatomical structures within the patient and other structures during use of guidewire (1000). Outer tube (1030) of the present example is also configured to not have an adverse impact on the signal provided by navigation coil (1010). In some versions, outer tube (1030) is constructed of a non-conductive polymeric material such as polyamide. In some other versions, outer tube (1030) is constructed of titanium, nitinol, 316 stainless steel, and/or some other material(s). Other suitable ways in which outer tube (1030) may be configured will be apparent to those of ordinary skill in the art in view of the teachings herein.

Solder joint (1020) is used to secure at least some of the above-described components together. In the present example, solder joint (1020) is proximal to navigation coil (1010) and extends about outer coil (1002), core wire (1008), and navigation cable (1012). Navigation coil (1010) proximally terminates distal to the longitudinal position of solder joint (1020). In addition to securing components of guidewire (1000) together, solder joint (1020) may also provide some degree of structural integrity to guidewire (1000). It should be understood that solder joint (1020) is merely optional such that components of guidewire (1000) may be secured together in any other suitable fashion.

By way of example only, outer coil (1002) may have an effective outer diameter of approximately 0.0315 inches and an inner diameter of approximately 0.0210 inches. Outer coil (1002) may also be formed by a 316 stainless steel (or nitinol) wire having a flat cross-sectional profile that is approximately 0.005 inches by approximately 0.007 inches. Navigation coil (1010) may have a length of approximately 0.059 inches and an effective outer diameter of approximately 0.031 inches. Core (1050) may have an outer diameter of approximately 0.015 inches. Outer tube (1030) may have an outer diameter of approximately 0.036 inches. Of course, all of these dimensions are just merely illustrative examples. Other suitable dimensions will be apparent to those of ordinary skill in the art in view of the teachings herein

VI. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) an outer coil having a distal end, wherein the outer coil defines an interior region; (b) a distal tip member secured to the distal end of the outer coil; and (c) a navigation coil, wherein a proximal portion of the navigation coil is located within the interior region of the outer coil, wherein a distal portion of the navigation coil is located in the distal tip member, wherein the navigation coil is configured to generate a signal in response to movement of the navigation coil within an electromagnetic field.

### Example 2

The apparatus of Example 1, wherein the distal tip member is formed of an optically transmissive material.

### Example 3

The apparatus of any one or more of Examples 1 through 2, further comprising an optical fiber extending longitudinally through the interior region of the outer coil.

### Example 4

The apparatus of Example 3, wherein the optical fiber is in optical communication with the distal tip member.

### Example 5

The apparatus of any one or more of Examples 3 through 4, wherein the navigation coil defines an effective outer diameter, wherein the optical fiber is positioned outside the effective outer diameter defined by the navigation coil.

### Example 6

The apparatus of any one or more of Examples 1 through 5, further comprising a core wire extending longitudinally through the interior region of the outer coil.

### Example 7

The apparatus of Example 6, wherein the navigation coil defines an effective outer diameter, wherein the core wire is positioned outside the effective outer diameter defined by the navigation coil.

### Example 8

An apparatus comprising: (a) an outer coil having a distal end, wherein the outer coil defines an interior region; (b) a distal tip member secured to the distal end of the outer coil; and (c) a navigation coil, wherein the navigation coil is located in the distal tip member at a position distal to the distal end of the outer coil, wherein the navigation coil is configured to generate a signal in response to movement of the navigation coil within an electromagnetic field.

### Example 9

The apparatus of Example 8, wherein the outer coil defines an inner diameter surrounding the interior region, wherein the navigation coil defines an effective outer diameter, wherein the effective outer diameter of the navigation coil is larger than the inner diameter of the outer coil.

### Example 10

The apparatus of any of the preceding or following Examples 8 through 9, further comprising a support tube, wherein at least a portion of the support tube is positioned within the navigation coil.

### Example 11

The apparatus of Example 10, wherein the support tube has a proximal end, wherein the navigation coil has a proximal end, wherein the proximal end of the support tube is proximal to the proximal end of the navigation coil.

### Example 12

The apparatus of any one or more of Examples 10 through 11, wherein the support tube has a cylindraceous shape.

### Example 13

The apparatus of any one or more of Examples 8 through 9, further comprising a support tube, wherein at least a portion of the support tube is positioned around the navigation coil.

### Example 14

The apparatus of Example 13, wherein the support tube comprises a proximal portion and a distal portion, wherein the proximal portion has a first inner diameter and a first outer diameter, wherein the distal portion has a second inner diameter and a second outer diameter, wherein the second inner diameter is larger than the first inner diameter, wherein the second outer diameter is larger than the second inner diameter.

### Example 15

The apparatus of Example 14, wherein the proximal portion is positioned within the interior region of the outer coil, wherein the distal portion is positioned about the navigation coil.

### Example 16

The apparatus of any one or more of Examples 10 through 15, further comprising a core wire, wherein the core wire extends through the support tube and into the distal tip member.

### Example 17

The apparatus of Example 16, wherein the core wire also extends through the navigation coil.

### Example 18

An apparatus comprising: (a) an outer extrusion having a distal end, wherein the outer extrusion defines an interior region; (b) a distal tip member secured to the distal end of the outer extrusion; and (c) a navigation coil, wherein a proximal portion of the navigation coil is located within the interior region of the outer coil, wherein a distal portion of the navigation coil is located in the distal tip member, wherein the navigation coil is configured to generate a signal in response to movement of the navigation coil within an electromagnetic field.

### Example 19

An apparatus comprising: (a) an outer coil having a distal end, wherein the outer coil defines an interior region; (b) a distal tip member secured to the distal end of the outer coil; (c) a navigation coil, wherein the navigation coil is located within the interior region of the outer coil, wherein at least a portion of the navigation coil is located proximal to the distal tip member, wherein the navigation coil is configured to generate a signal in response to movement of the navigation coil within an electromagnetic field, wherein the navigation coil defines an inner diameter; and (d) a ferromagnetic core located within the inner diameter of the navigation coil.

### Example 20

An apparatus comprising: (a) an outer coil having a distal end, wherein the outer coil defines an interior region bounded by inner diameter; (b) a distal tip member secured to the distal end of the outer coil; (c) a navigation coil, wherein the navigation coil is located within the interior region of the outer coil, wherein at least a portion of the navigation coil is located proximal to the distal tip member, wherein the navigation coil is configured to generate a signal in response to movement of the navigation coil within an electromagnetic field, wherein the navigation coil defines an outer diameter; and (d) a support tube interposed between the inner diameter of the outer coil and the outer diameter of the navigation coil.

### Example 21

The apparatus of Example 20, wherein the support tube is adhered to the outer diameter of the navigation coil.

### Example 22

The apparatus of any one or more of Examples 20 through 21, wherein the support tube is adhered to the inner diameter of the outer coil.

### Example 23

An apparatus comprising: (a) an outer coil having a distal end, wherein the outer coil defines an interior region bounded by inner diameter; (b) a navigation coil, wherein the navigation coil is positioned distal to the distal end of the outer coil, wherein the navigation coil is configured to generate a signal in response to movement of the navigation coil within an electromagnetic field; and (c) a distal tip member positioned distal to the navigation coil, such that the navigation coil is longitudinally interposed between the distal tip member and the distal end of the outer coil.

### Example 24

The apparatus of Example 23, wherein the navigation coil defines an effective diameter that is larger than the inner diameter of the outer coil.

### Example 25

The apparatus of any one or more of Examples 23 through 24, further comprising an outer tube positioned about the navigation coil.

### Example 26

The apparatus of Example 25, wherein the outer tube has a proximal end, wherein the proximal end of the outer tube is secured to the distal end of the outer coil.

### Example 27

The apparatus of any one or more of Examples 25 through 26, wherein the outer tube has a distal end, wherein the distal end of the outer tube is secured to the distal tip member.

### Example 28

The apparatus of any one or more of Examples 25 through 27, wherein the outer tube is adhered to the navigation coil.

### Example 29

The apparatus of any one or more of Examples 25 through 28, wherein the outer tube comprises polyamide.

### Example 30

The apparatus of any one or more of Examples 23 through 29, wherein the navigation coil proximally terminates at a proximal end, wherein the proximal end of the navigation coil is distal to the distal end of the outer coil.

### Example 31

The apparatus of any one or more of Examples 23 through 30, wherein the navigation coil distally terminates at a distal end, wherein the distal end of the navigation coil is proximal to the distal tip member.

### Example 32

The apparatus of any one or more of Examples 23 through 31, further comprising a ferrous core, wherein the ferrous core is positioned within an interior defined by the navigation coil.

### Example 33

The apparatus of any one or more of Examples 23 through 32, further comprising an electrical wire coupled with the navigation coil, wherein the electrical wire extends through the interior region of the outer coil.

### Example 34

The apparatus of any one or more of Examples 23 through 33, further comprising a core wire extending through the interior region of the outer coil, wherein a distal end of the core wire is secured to the outer coil.

### Example 35

The apparatus of Example 34, wherein the distal end of the core wire is secured to the outer coil by solder forming a solder joint.

### Example 36

The apparatus of Example 35, further comprising an outer tube positioned about the navigation coil, wherein the outer tube has a proximal end secured to the solder joint.

### Example 37

The apparatus of any one or more of Examples 23 through 36, further comprising a navigation system, wherein the navigation system is operable to generate an electromagnetic field, wherein the navigation coil is configured to generate a signal in response to movement of the navigation coil within the electromagnetic field.

### Example 38

An apparatus comprising: (a) an outer coil having a distal end, wherein the outer coil defines an interior region; (b) a distal tip member secured relative to the distal end of the outer coil; and (c) a navigation coil, wherein the navigation coil is located at a position distal to the distal end of the outer coil, wherein the navigation coil is configured to generate a signal in response to movement of the navigation coil within an electromagnetic field.

### Example 39

The apparatus of Example 38, wherein the navigation coil is longitudinally interposed between the distal tip member and the distal end of the outer coil.

### Example 40

The apparatus of any one or more of Examples 38 through 39, wherein the navigation coil is located in the distal tip member.

### Example 41

An apparatus comprising: (a) an outer coil having a distal end, wherein the outer coil defines an interior region; (b) a distal tip member secured relative to the distal end of the outer coil; (c) a navigation coil, wherein at least a portion of the navigation coil is located proximal to the distal tip member, wherein at least a portion of the navigation coil is located distal to the distal end of the outer coil, wherein the navigation coil is configured to generate a signal in response to movement of the navigation coil within an electromagnetic field, wherein the navigation coil defines an inner diameter; and (d) a ferromagnetic core located within the inner diameter of the navigation coil.

### Example 42

The apparatus of Example 41, wherein the navigation coil defines a length, wherein the entire length of the navigation coil is positioned between the distal tip member and the distal end of the outer coil.

VII. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) an outer coil having a distal end, wherein the outer coil defines an interior region bounded by inner diameter;
(b) a navigation coil, wherein the navigation coil is positioned distal to the distal end of the outer coil, wherein the navigation coil is configured to generate a signal in response to movement of the navigation coil within an electromagnetic field; and
(c) a distal tip member positioned distal to the navigation coil, such that the navigation coil is longitudinally interposed between the distal tip member and the distal end of the outer coil.

2. The apparatus of claim 1, wherein the navigation coil defines an effective diameter that is larger than the inner diameter of the outer coil.

3. The apparatus of claim 1, further comprising an outer tube positioned about the navigation coil.

4. The apparatus of claim 3, wherein the outer tube has a proximal end, wherein the proximal end of the outer tube is secured to the distal end of the outer coil.

5. The apparatus of claim 3, wherein the outer tube has a distal end, wherein the distal end of the outer tube is secured to the distal tip member.

6. The apparatus of claim 3, wherein the outer tube is adhered to the navigation coil.

7. The apparatus of claim 3, wherein the outer tube comprises polyamide.

8. The apparatus of claim 1, wherein the navigation coil proximally terminates at a proximal end, wherein the proximal end of the navigation coil is distal to the distal end of the outer coil.

9. The apparatus of claim 1, wherein the navigation coil distally terminates at a distal end, wherein the distal end of the navigation coil is proximal to the distal tip member.

10. The apparatus of claim 1, further comprising a ferrous core, wherein the ferrous core is positioned within an interior defined by the navigation coil.

11. The apparatus of claim 1, further comprising an electrical wire coupled with the navigation coil, wherein the electrical wire extends through the interior region of the outer coil.

12. The apparatus of claim 1, further comprising a core wire extending through the interior region of the outer coil, wherein a distal end of the core wire is secured to the outer coil.

13. The apparatus of claim 12, wherein the distal end of the core wire is secured to the outer coil by solder forming a solder joint.

14. The apparatus of claim 13, further comprising an outer tube positioned about the navigation coil, wherein the outer tube has a proximal end secured to the solder joint.

15. The apparatus of claim 1, further comprising a navigation system, wherein the navigation system is operable to generate an electromagnetic field, wherein the navigation coil is configured to generate a signal in response to movement of the navigation coil within the electromagnetic field.

16. An apparatus comprising:
(a) an outer coil having a distal end, wherein the outer coil defines an interior region;
(b) a distal tip member secured relative to the distal end of the outer coil; and
(c) a navigation coil, wherein the navigation coil is located at a position distal to the distal end of the outer coil, wherein the navigation coil is configured to generate a signal in response to movement of the navigation coil within an electromagnetic field.

17. The apparatus of claim 16, wherein the navigation coil is longitudinally interposed between the distal tip member and the distal end of the outer coil.

18. The apparatus of claim 16, wherein the navigation coil is located in the distal tip member.

19. An apparatus comprising:
(a) an outer coil having a distal end, wherein the outer coil defines an interior region;
(b) a distal tip member secured relative to the distal end of the outer coil;
(c) a navigation coil, wherein at least a portion of the navigation coil is located proximal to the distal tip member, wherein at least a portion of the navigation coil is located distal to the distal end of the outer coil, wherein the navigation coil is configured to generate a signal in response to movement of the navigation coil within an electromagnetic field, wherein the navigation coil defines an inner diameter; and
(d) a ferromagnetic core located within the inner diameter of the navigation coil.

20. The apparatus of claim 19, wherein the navigation coil defines a length, wherein the entire length of the navigation coil is positioned between the distal tip member and the distal end of the outer coil.
